(19) United States
(12) Patent Application Publication
(10) Pub. No.: US 2021/0316065 A1
(43) Pub. Date: Oct. 14, 2021

(54) SYRINGE COLLAR AND FRAME

(71) Applicant: BAYER HEALTHCARE LLC, WHIPPANY, NJ (US)

(72) Inventors: David Berry, Kittanning, PA (US); Joseph Ranalletta, Greenville, SC (US); Doreen Weightman, Oakdale, PA (US); Christos Sarantos, Lakeland, FL (US); Thomas R. Massey, II, Knoxville, TN (US); Kevin Cowan, Allison Park, PA (US); Patrick Campbell, Pittsburgh, PA (US); Barry Tucker, Verona, PA (US); Gerald Callan, Cranberry Twp., PA (US)

(21) Appl. No.: 17/274,268

(22) PCT Filed: Sep. 10, 2019
(86) PCT No.: PCT/US2019/050340

§ 371 Date: Mar. 8, 2021

Related U.S. Application Data
(60) Provisional application No. 62/730,153, filed on Sep. 12, 2018.
(60) Provisional application No. 62/831,004, filed on Apr. 8, 2019.

(51) Int. Cl. A61M 5/145; A61M 5/315

(52) U.S. Cl. CPC A61M 5/14546; A61M 2005/3128; A61M 2005/14553; A61M 5/31596

(57) ABSTRACT

A frame for engaging at least two fluid containers includes: a first engagement member configured to secure at least a portion of one of the at least two fluid containers; a second engagement member configured to secure at least a portion of another one of the at least two fluid containers; and a body structure connected to the first engagement member and the second engagement member. The body structure is sized and shaped to index the at least two fluid containers with a powered injector system such that the at least two fluid injectors are held in an orientation and distance relative to each other for insertion into at least two pressure jackets associated with the powered injector system.

### CROSS REFERENCE TO RELATED APPLICATIONS

This application claims priority to U.S. Provisional Patent Application Ser. No. 62/730,153, entitled “Syringe Collar and Frame”, filed Sep. 12, 2018, and U.S. Provisional Patent Application Ser. No. 62/831,004, filed Apr. 8, 2019, the contents of which are incorporated herein by reference.

### BACKGROUND OF THE DISCLOSURE

### Field

The present disclosure relates to syringe collar and frame elements for managing the syringe arrangements and patient tubing and filling components for ease of use in the preparation of a fluid injector for a fluid injection procedure.

### Description of Related Art

Contrast media are administered to patients to enhance the contrast of bodily structures or fluids during certain medical procedures. For example, contrast media are used in diagnostic imaging procedures, including X-ray, computed tomography (CT), magnetic resonance imaging (MRI), cardiovascular angiography (CV), and ultrasound, and in interventional radiological procedures, such as angioplasty and certain types of chemotherapy. Various forms and concentrations of contrast media are available and are selected based on the type of procedure and the subject of interest. Illustrative contrast media include gadolinium-, barium- and iodine-based solutions commonly used in radiological imaging procedures. Contrast media may be delivered to patients through various methods, including ingestion, manual injections, or automated fluid delivery systems.

In many medical diagnostic and therapeutic procedures, a physician or other person injects a patient with a fluid. In recent years, a number of injector-actuated syringes and fluid injectors for pressurized injection of medical fluids, such as a contrast solution (often referred to simply as “contrast”), a flushing agent, such as saline, and other medical fluids, have been developed for use in procedures such as angiography, computed tomography (CT), ultrasound, magnetic resonance imaging (MRI), positron emission tomography (PET), and other molecular imaging procedures. In general, these fluid injectors are designed to deliver a preset amount of fluid at a preset pressure and/or flow rate.

Typically, such powered injectors include a housing allowing two syringes to be connected to a front wall thereof. These injectors further comprise drive members such as pistons that connect to plungers or an engagement feature on a proximal end wall of the syringes. Syringes used with a front-loading injector typically include a readily releasable mounting mechanism for securing the syringe to the front wall of the injector. Such syringes may, for example, include a rigid syringe body, a plunger reciprocally mounted therein, and a plunger extension for transfer of force to the plunger. In other embodiments, the syringe may include a rolling diaphragm barrel configuration having a flexible sidewall, where the proximal end wall of the syringe body releasably engages and interacts with the at least one drive member. The drive members drive the plungers of the syringe or the rolling diaphragm proximal end wall in a proximal and/or distal direction relative to a longitudinal axis of the barrel to draw fluid into the syringe barrel or deliver the fluid from the syringe barrel.

In a typical procedure, a first syringe is mounted, by a user, to the front wall of the powered injector utilizing the releasable mounting mechanism. Thereafter, a second syringe is mounted, by a user, to the front wall of the powered injector utilizing the releasable mounting mechanism. Then, suitable filling components such as tubing and spikes are mounted to the syringes and the syringes are filled with the appropriate fluids for a specific injection procedure. The filling components are then removed and patient tubing is coupled to the syringes, the tubing is primed and connected to a patient, and a fluid injection procedure is initiated.

In many medical applications, however, powered injectors are used in procedures and areas in which there are substantial time and access constraints. In time and/or access constrained procedures, loading each of the individual syringes into the injector, properly aligning each of the individual syringes, and properly locking each of the individual syringes to the injector, results in inefficiencies that can be expedited.

Accordingly, a need exists for a system that indexes the syringes within the injector so that they are held in the proper orientation and distance for insertion into the injector, and allows a user to quickly and easily install and remove the syringes within the injector, thereby, improving workflow by requiring fewer steps for setup of the injection procedure while maintaining sterility of the various components.

### SUMMARY OF THE DISCLOSURE

The present disclosure relates to a system that indexes syringes within an injector so that they are held in the proper orientation and distance for insertion into the injector, and allows a user to quickly and easily install and remove the syringes within the injector, thereby, improving workflow by requiring fewer steps for setup of the injection procedure while maintaining sterility of the various components.

In one example, provided is a frame for engaging at least two fluid containers. The frame comprises: a first engagement member configured to secure at least a portion of one of the at least two fluid containers; a second engagement member configured to secure at least a portion of another one of the at least two fluid containers; and a body structure connected to the first engagement member and the second engagement member. The body structure is sized and shaped to index the at least two fluid containers with a powered injector system such that the at least two fluid containers are held in an orientation and distance relative to each other for insertion into the powered injector system.

In some examples, the at least two fluid containers may be syringes, such as rolling diaphragm syringes.

In some examples, the first engagement member and the second engagement member may be generally C-shaped and are configured to engage a retention flange provided on a distal end of each of the fluid container. The first engagement member and the second engagement member may each comprise a notch configured to accommodate a valve assembly positioned over a distal end of the fluid container. The body structure may comprise a first leg having a first end extending vertically from the first engagement member and a second end, a second leg having a first end extending vertically from the second engagement member and a second end, and a body member connected between the second end of the first leg and the second end of the second leg. The lengths of the first leg and the second leg may be selected to provide a proper vertical orientation of the at least two fluid containers when positioned within the powered injector system. The body structure may be configured as a handle for carrying the at least two fluid containers.

In some examples, the frame may further comprise a support structure extending from the body structure. The support structure may be configured to secure a valve assembly in fluid communication with outlets of the at least two fluid containers, the valve assembly configured to deliver fluid to a patient fluid tubing set from one of the at least two fluid containers while preventing fluid from being delivered to the patient fluid tubing set from others of the at least two fluid containers or to deliver a selected ratio of at least two fluids to a patient fluid tubing set from the at least two fluid containers when a motive force is applied to each piston of the powered injector system associated with the at least two fluid containers. The support structure may be configured to support and manage at least one of: tubing used to fill the at least two fluid containers; spikes used to connect the at least two fluid containers to a bulk fluid source; patient tubing; a priming tube; or any combination thereof.

In some examples, the support structure may comprise an oval portion configured to manage at least one of tubing used to fill the at least two fluid containers and spikes used to connect the at least two fluid containers to a bulk fluid source. The oval portion may comprise clips on a top surface thereof configured to secure at least a portion of the patient tubing set to the oval portion. The support structure may be molded as a first half and second half with the first half and the second half are connected to the body structure via a hinge mechanism such that the first half and the second half can be connected together to form the support structure by closing the hinge mechanism. In some examples, the frame may further comprise a clamping structure having a first end configured to engage the body structure and a second configured to secure a valve assembly.

In one example, the first engagement structure may engage a fluid outlet end of one of the at least two fluid containers and the second engagement structure may engage a fluid outlet end of another one of the at least two fluid containers in a manner to allow limited proximal and distal movement of the at least two fluid containers during a fluid filling and a fluid injection procedure, respectively. In another example, the first engagement structure engages a fluid outlet end of one of the at least two fluid containers and the second engagement structure engages a fluid outlet end of another one of the at least two fluid containers in a manner to prevent proximal and distal movement of the at least two fluid containers during a fluid filling and a fluid injection procedure.

The body structure may further comprise at least one spike in fluid communication with at least one of the at least two fluid containers for connection of at least one bulk fluid source with the at least one of the at least two fluid containers. The at least one spike may be provided in fluid communication with at least one of the at least two fluid containers via at least one of tubing or a manifold associated with the frame.

In some examples, the first engagement member and the second engagement member may configured as a first pressure jacket cap and a second pressure jacket cap, respectively, each comprising a frusto-conical structure that engages and surrounds a frusto-conical distal end of each of the at least two fluid containers. Alternatively, the first engagement member may comprise a first syringe cap and the second engagement member may comprise a second syringe cap. The first syringe cap and a first portion of the body structure may be connected to the second syringe cap and a second portion of the body structure by an engagement mechanism provided between the first portion of the body structure and the second portion of the body structure. The engagement mechanism may permanently connect the first portion of the body structure and the second portion of the body structure or removably connect the first portion of the body structure and the second portion of the body structure. The first portion of the body structure and the second portion of the body structure may be color coded according to the type of fluid to be used in each of the at least two fluid containers.

In some examples, the frame may further comprise at least one clip structure connected to and extending from the body structure. The at least one clip structure may comprise a first clip structure and a first clip extending from a first side of the body structure for securing a first spike, provided in fluid communication with at least one of the at least two fluid containers, thereto and a second clip structure and a second clip extending from a second side of the body structure for securing a bulk fluid source thereto. The first clip may be hingedly connected to the second clip such that, after the bulk fluid source is removed from the second clip, the first clip and the spike are rotated relative to the second clip to provide the spike in an exposed position and the bulk fluid source is connected to the spike to fill at least one of the at least two fluid containers. Alternatively, the first clip may be hingedly connected to the second clip such that, after the bulk fluid source is removed from the second clip and reattached to the second clip in an inverted position, the first clip and the spike are moved relative to the second clip to engage the spike with the bulk fluid source to fill at least one of the at least two fluid containers. In yet another example, the first clip may be hingedly connected to the second clip such that, after the spike is removed from the first clip and provided in fluid communication with the bulk fluid source, the second clip and the bulk fluid source are moved relative to the first clip to provide the bulk fluid source in an inverted position to fill at least one of the at least two fluid containers.

In still another example, the clip structure may comprise a first clip extending from a first side of the body structure for securing a first spike provided in fluid communication with one of the at least two fluid containers and a second spike provided in fluid communication with another of the at least two fluid containers and a second clip extending from a second side of the body structure for securing a first bulk fluid source thereto. The first spike may be configured to be removed from the first clip and fluidly connected to the first bulk fluid source and the first bulk fluid source may be configured to be removed from the second clip and reattached to the second clip in an inverted position after the first spike has been attached thereto to fill the one of the at least two fluid containers. The second spike may be configured to be removed from the first clip and fluidly connected to a second bulk fluid source to fill another of the least at two fluid containers.

In some examples, the first engagement member, the second engagement member, the body structure, and the at least two fluid containers may be integrally formed. In another example, the body structure may comprise at least one identification component provided thereon configured to be read by the powered injector system to provide information to the powered injector system. The information may comprise at least one of information related to the at least two fluid containers, information related to an injection procedure, information related to the patient, or any combination thereof.

In accordance with another aspect of the present disclosure, provided is a syringe system for a powered injector system. The syringe system comprises: a first syringe; a second syringe; and a frame for engaging the first syringe and the second syringe. The frame comprises: a first engagement member configured to secure at least a portion of the first syringe; a second engagement member configured to secure at least a portion of the second syringe; and a body structure connected to the first engagement member and the second engagement member. The body structure is sized and shaped to index the first syringe and the second syringe with the powered injector system such that the first syringe and second syringe are held in an orientation and distance relative to each other for insertion into the powered injector system. In some examples, the first syringe and the second syringe may be rolling diaphragm syringes.

The first engagement member and the second engagement member may be generally C-shaped and are configured to engage a flange provided on a distal end of each of the first and second syringes. In addition, the first engagement member and the second engagement member may each comprise a notch configured to accommodate a valve assembly positioned over a distal end of the first syringe and the second syringe. The body structure may comprise a first leg having a first end extending vertically from the first engagement member and a second end, a second leg having a first end extending vertically from the second engagement member and a second end, and a body member connected between the second end of the first leg and the second end of the second leg. The lengths of the first leg and the second leg may be selected to provide a proper vertical orientation of the first syringe and second syringe when positioned within the powered injector system. In addition, the body structure may be configured as a handle for carrying the first syringe and the second syringe.

In some examples the syringe system may further comprise a support structure extending from the body structure. The support structure may be configured to secure a valve assembly in fluid communication with outlets of the first syringe and the second syringe. The valve assembly may be configured to deliver fluid to a patient fluid tubing set from one of the first syringe and the second syringe while preventing fluid from being delivered to the patient fluid tubing set from the other of the first syringe and the second syringe or to deliver a selected ratio of at least two fluids to a patient fluid tubing set from the at least two fluid containers when a motive force is applied to each piston of the powered injector system associated with the at least two fluid containers. The support structure may also be configured to support and manage at least one of: tubing used to fill the first syringe and the second syringe; spikes used to connect the first syringe and the second syringe to a bulk fluid source; patient tubing; a priming tube; or any combination thereof.

In some examples, the support structure may comprise an oval portion configured to manage at least one of tubing used to fill the first syringe and the second syringe and spikes used to connect the first syringe and the second syringe to a bulk fluid source. The oval portion may comprise clips on a top surface thereof configured to secure at least a portion of the patient tubing set to the oval portion. The support structure may be molded as a first half and second half with the first half and the second half are connected to the body structure via a hinge mechanism such that the first half and the second half can be connected together to form the support structure by closing the hinge mechanism. In another example, a clamping structure having a first end configured to engage the body structure and a second may be configured to secure a valve assembly.

The first engagement structure may engage a fluid outlet end of one of the first syringe and the second syringe and the second engagement structure may engage a fluid outlet end of the other of the first syringe and the second syringe in a manner to allow limited proximal and distal movement of the first syringe and the second syringe during a fluid filing and a fluid injection procedure, respectively. Alternatively, the first engagement structure may engage a fluid outlet end of one of the first syringe and the second syringe and the second engagement structure may engage a fluid outlet end of the other of the first syringe and the second syringe in a manner to prevent proximal and distal movement of the first syringe and the second syringe during a fluid filling and fluid injection procedure.

In some examples, the body structure may further comprise at least one spike in fluid communication with at least one of the first syringe and the second syringe for connection of at least one bulk fluid source with the at least one of the first syringe and the second syringe. The at least one spike may be provided in fluid communication with at least one of the first syringe and the second syringe via at least one of tubing or a manifold associated with the frame.

In one example, the first engagement member and the second engagement member may be configured as a first pressure jacket cap and a second pressure jacket cap, respectively, each comprising a frusto-conical structure that engages and surrounds a frusto-conical distal end of each of the first syringe and the second syringe. Alternatively, the first engagement member may comprise a first syringe cap and the second engagement member may comprise a second syringe cap. The first syringe cap and a first portion of the body structure may be connected to the second syringe cap and a second portion of the body structure by an engagement mechanism provided between the first portion of the body structure and the second portion of the body structure. The engagement mechanism may permanently connect the first portion of the body structure and the second portion of the body structure or removably connect the first portion of the body structure and the second portion of the body structure. The first portion of the body structure and the second portion of the body structure may be color coded according to the type of fluid to be used in the first and second syringes.

In one example, the first engagement member, the second engagement member, the body structure, the first syringe, and the second syringe may be integrally formed. The body structure may comprise at least one identification component provided thereon configured to be read by the powered injector system to provide information to the powered injector system. The information may comprise at least one of information related to the first syringe and the second syringe, information related to an injection procedure, information related to the patient, or any combination thereof.

In accordance with another aspect of the present disclosure, provided is a syringe for a powered injector system. The syringe comprises: a proximal end, a distal end, and a cylindrical sidewall between the proximal end and the distal end defining an interior volume for retaining a medical fluid therein; a discharge nozzle at the distal end; a piston engagement feature located on one of plunger slidably associated with the syringe and a proximal end wall of the syringe. The piston engagement feature is configured for releasably engaging a piston of the powered injector system; and a frame positioned at the distal end of the syringe. The frame is configured to index the syringe with the power injector system such that the syringe is held in a proper vertical position within the powered injector system for conducting a fluid injection procedure.

In some examples, the frame may comprise a support structure extending therefrom. The support structure may be configured to support and manage at least one of: tubing used to fill the syringe; spikes used to connect the syringe to a bulk fluid source; a patient tubing set; a priming tube; and any combination thereof. The support structure may comprise an oval portion configured to manage at least one of at least one tubing used to fill the syringe and at least one spike used to connect the syringe to a bulk fluid source. The oval portion may comprise clips on a top surface thereof configured to secure at least a portion of the patient tubing set to the oval portion. The support structure may be molded as a first half and second half with the first half and the second half are connected to the frame via a hinge mechanism such that the first half and the second half can be connected together to form the support structure by closing the hinge mechanism.

In some examples, the frame may comprise at least one spike in fluid communication with the syringe for fluidly connecting of at least one bulk fluid source with the syringe. The at least one spike may be provided in fluid communication with the syringe via at least one of tubing or a manifold associated with the frame.

The frame may comprise an engagement mechanism for connecting the frame to a second frame of a second syringe. The connected frame and second frame may be configured to index the syringe and the second syringe with the power injector system such that the syringe and second syringe are held in a proper vertical position within the powered injector system for conducting a fluid injection procedure. The engagement mechanism may permanently connect the frame to the second frame of the second syringe or removably connects the frame to the second frame of the second syringe. In some examples, the frame and the syringe may be integrally formed. The frame may comprise at least one identification component provided thereon configured to be read by the powered injector system to provide information to the powered injector system. The information may comprise at least one of information related to the syringe, information related to an injection procedure, information related to the patient, or any combination thereof.

Various aspects of the system and method for injector position calibration of the fluid injector are disclosed in one or more of the following numbered clauses:

Clause 1: A frame for engaging at least two fluid containers, the frame comprising: a first engagement member configured to secure at least a portion of one of the at least two fluid containers; a second engagement member configured to secure at least a portion of another one of the at least two fluid containers; and a body structure connected to the first engagement member and the second engagement member, wherein the body structure is sized and shaped to index the at least two fluid containers with a powered injector system such that the at least two fluid containers are held in an orientation and distance relative to each other for insertion into the powered injector system.

Clause 2. The frame of clause 1, wherein the at least two fluid containers are syringes.

Clause 3. The frame of clause 2, wherein the at least two fluid containers are rolling diaphragm syringes.

Clause 4. The frame of any of clauses 1 to 3, wherein the first engagement member and the second engagement member are generally C-shaped and are configured to engage a retention flange provided on a distal end of each of the fluid container.

Clause 5. The frame of any of clauses 1 to 4, wherein the first engagement member and the second engagement member each comprise a notch configured to accommodate a valve assembly positioned over a distal end of the fluid container.

Clause 6. The frame of any of clauses 1 to 5, wherein the body structure comprises a first leg having a first end extending vertically from the first engagement member and a second end, a second leg having a first end extending vertically from the second engagement member and a second end, and a body member connected between the second end of the first leg and the second end of the second leg.

Clause 7. The frame of clause 6, wherein lengths of the first leg and the second leg are selected to provide a proper vertical orientation of the at least two fluid containers when positioned within the powered injector system.

Clause 8. The frame of any of clauses 1 to 7, wherein the body structure is configured as a handle for carrying the at least two fluid containers.

Clause 9. The frame of any of clauses 1 to 8, further comprising a support structure extending from the body structure.

Clause 10. The frame of clause 9, wherein the support structure is configured to secure a valve assembly in fluid communication with outlets of the at least two fluid containers, the valve assembly configured to deliver fluid to a patient fluid tubing set from one of the at least two fluid containers while preventing fluid from being delivered to the patient fluid tubing set from others of the at least two fluid containers or to deliver a selected ratio of at least two fluids to a patient fluid tubing set from the at least two fluid containers when a motive force is applied to each piston of the powered injector system associated with the at least two fluid containers.

Clause 11. The frame of clause 9 or 10, wherein the support structure is configured to support and manage at least one of: tubing used to fill the at least two fluid containers; spikes used to connect the at least two fluid containers to a bulk fluid source; patient tubing; a priming tube; or any combination thereof.

Clause 12. The frame of any of clauses 9 to 11, wherein the support structure comprises an oval portion configured to manage at least one of tubing used to fill the at least two fluid containers and spikes used to connect the at least two fluid containers to a bulk fluid source.

Clause 13. The frame of clause 12, wherein the oval portion comprises clips on a top surface thereof configured to secure at least a portion of the patient tubing set to the oval portion.

Clause 14. The frame of any of clauses 9 to 13, wherein the support structure is molded as a first half and second half with the first half and the second half are connected to the body structure via a hinge mechanism such that the first half and the second half can be connected together to form the support structure by closing the hinge mechanism.

Clause 15. The frame of any of clauses 1 to 8, further comprising a clamping structure having a first end configured to engage the body structure and a second configured to secure a valve assembly.

Clause 16. The frame of any of clauses 1 to 15, wherein the first engagement structure engages a fluid outlet end of one of the at least two fluid containers and the second engagement structure engages a fluid outlet end of another one of the at least two fluid containers in a manner to allow limited proximal and distal movement of the at least two fluid containers during a fluid filling and a fluid injection procedure, respectively.

Clause 17. The frame of any of clauses 1 to 15, wherein the first engagement structure engages a fluid outlet end of one of the at least two fluid containers and the second engagement structure engages a fluid outlet end of another one of the at least two fluid containers in a manner to prevent proximal and distal movement of the at least two fluid containers during a fluid filling and a fluid injection procedure.

Clause 18. The frame of any of clauses 1 to 17, wherein the body structure further comprises at least one spike in fluid communication with at least one of the at least two fluid containers for connection of at least one bulk fluid source with the at least one of the at least two fluid containers.

Clause 19. The frame of clause 18, wherein the at least one spike is provided in fluid communication with at least one of the at least two fluid containers via at least one of tubing or a manifold associated with the frame.

Clause 20. The frame of any of clauses 1 to 19, wherein the first engagement member and the second engagement member are configured as a first pressure jacket cap and a second pressure jacket cap, respectively, each comprising a frusto-conical structure that engages and surrounds a frusto-conical distal end of each of the at least two fluid containers.

Clause 21. The frame of any of clauses 1 to 19, wherein the first engagement member comprises a first syringe cap and the second engagement member comprises a second syringe cap, and wherein the first syringe cap and a first portion of the body structure is connected to the second syringe cap and a second portion of the body structure by an engagement mechanism provided between the first portion of the body structure and the second portion of the body structure.

Clause 22. The frame of clause 21, wherein the engagement mechanism permanently connects the first portion of the body structure and the second portion of the body structure or removably connects the first portion of the body structure and the second portion of the body structure.

Clause 23. The frame of clause 21 or 22, wherein the first portion of the body structure and the second portion of the body structure are color coded according to the type of fluid to be used in each of the at least two fluid containers.

Clause 24. The frame of any of clauses 1 to 23, further comprising at least one clip structure connected to and extending from the body structure.

Clause 25. The frame of clause 24, wherein the at least one clip structure comprises a first clip structure and a first clip extending from a first side of the body structure for securing a first spike, provided in fluid communication with at least one of the at least two fluid containers, thereto and a second clip structure and a second clip extending from a second side of the body structure for securing a bulk fluid source thereto.

Clause 26. The frame of clause 25, wherein the first clip is hingedly connected to the second clip such that, after the bulk fluid source is removed from the second clip, the first clip and the spike are rotated relative to the second clip to provide the spike in an exposed position and the bulk fluid source is connected to the spike to fill at least one of the at least two fluid containers.

Clause 27. The frame of clause 25, wherein the first clip is hingedly connected to the second clip such that, after the bulk fluid source is removed from the second clip and reattached to the second clip in an inverted position, the first clip and the spike are moved relative to the second clip to engage the spike with the bulk fluid source to fill at least one of the at least two fluid containers.

Clause 28. The frame of clause 25, wherein the first clip is hingedly connected to the second clip such that, after the spike is removed from the first clip and provided in fluid communication with the bulk fluid source, the second clip and the bulk fluid source are moved relative to the first clip to provide the bulk fluid source in an inverted position to fill at least one of the at least two fluid containers.

Clause 29. The frame of clause 24, wherein the clip structure comprises a first clip extending from a first side of the body structure for securing a first spike provided in fluid communication with one of the at least two fluid containers and a second spike provided in fluid communication with another of the at least two fluid containers and a second clip extending from a second side of the body structure for securing a first bulk fluid source thereto.

Clause 30. The frame of clause 29, wherein the first spike is configured to be removed from the first clip and fluidly connected to the first bulk fluid source and the first bulk fluid source is configured to be removed from the second clip and reattached to the second clip in an inverted position after the first spike has been attached thereto to fill the one of the at least two fluid containers.

Clause 31. The frame of clause 30, wherein the second spike is configured to be removed from the first clip and fluidly connected to a second bulk fluid source to fill another of the least at two fluid containers.

Clause 32. The frame of any of clauses 1 to 31, wherein the first engagement member, the second engagement member, the body structure, and the at least two fluid containers are integrally formed.

Clause 33. The frame of any of clauses 1 to 32, wherein the body structure comprises at least one identification component provided thereon configured to be read by the powered injector system to provide information to the powered injector system.

Clause 34. The frame of clause 33, wherein the information comprises at least one of information related to the at least two fluid containers, information related to an injection procedure, information related to the patient, or any combination thereof.

Clause 35. A syringe system for a powered injector system, the syringe system comprising: a first syringe; a second syringe; and a frame for engaging the first syringe and the second syringe, the frame comprising: a first engagement member configured to secure at least a portion of the first syringe; a second engagement member configured to secure at least a portion of the second syringe; and a body structure connected to the first engagement member and the second engagement member, wherein the body structure is sized and shaped to index the first syringe and the second syringe with the powered injector system such that the first syringe and second syringe are held in an orientation and distance relative to each other for insertion into the powered injector system.

Clause 36. The syringe system of clause 35, wherein the first syringe and the second syringe are rolling diaphragm syringes.

Clause 37. The syringe system of clause 35 or 36, wherein the first engagement member and the second engagement member are generally C-shaped and are configured to engage a flange provided on a distal end of each of the first and second syringes.

Clause 38. The syringe system of any of clauses 35 to 37, wherein the first engagement member and the second engagement member each comprise a notch configured to accommodate a valve assembly positioned over a distal end of the first syringe and the second syringe.

Clause 39. The syringe system of any of clauses 35 to 38, wherein the body structure comprises a first leg having a first end extending vertically from the first engagement member and a second end, a second leg having a first end extending vertically from the second engagement member and a second end, and a body member connected between the second end of the first leg and the second end of the second leg.

Clause 40. The syringe system of clause 39, wherein lengths of the first leg and the second leg are selected to provide a proper vertical orientation of the first syringe and second syringe when positioned within the powered injector system.

Clause 41. The syringe system of any of clauses 35 to 40, wherein the body structure is configured as a handle for carrying the first syringe and the second syringe.

Clause 42. The syringe system of any of clauses 35 to 41, further comprising a support structure extending from the body structure.

Clause 43. The syringe system of clause 42, wherein the support structure is configured to secure a valve assembly in fluid communication with outlets of the first syringe and the second syringe, the valve assembly configured to deliver fluid to a patient fluid tubing set from one of the first syringe and the second syringe while preventing fluid from being delivered to the patient fluid tubing set from the other of the first syringe and the second syringe or to deliver a selected ratio of at least two fluids to a patient fluid tubing set from the at least two fluid containers when a motive force is applied to each piston of the powered injector system associated with the at least two fluid containers.

Clause 44. The syringe system of clause 42 or 43, wherein the support structure is configured to support and manage at least one of: tubing used to fill the first syringe and the second syringe; spikes used to connect the first syringe and the second syringe to a bulk fluid source; patient tubing; a priming tube; or any combination thereof.

Clause 45. The syringe system of any of clauses 42 to 44, wherein the support structure comprises an oval portion configured to manage at least one of tubing used to fill the first syringe and the second syringe and spikes used to connect the first syringe and the second syringe to a bulk fluid source.

Clause 46. The syringe system of clause 45, wherein the oval portion comprises clips on a top surface thereof configured to secure at least a portion of the patient tubing set to the oval portion.

Clause 47. The syringe system of any of clauses 42 to 46, wherein the support structure is molded as a first half and second half with the first half and the second half are connected to the body structure via a hinge mechanism such that the first half and the second half can be connected together to form the support structure by closing the hinge mechanism.

Clause 48. The syringe system of any of clauses 35 to 41, further comprising a clamping structure having a first end configured to engage the body structure and a second configured to secure a valve assembly.

Clause 49. The syringe system of any of clauses 35 to 48, wherein the first engagement structure engages a fluid outlet end of one of the first syringe and the second syringe and the second engagement structure engages a fluid outlet end of the other of the first syringe and the second syringe in a manner to allow limited proximal and distal movement of the first syringe and the second syringe during a fluid filing and a fluid injection procedure, respectively.

Clause 50. The syringe system of any of clauses 35 to 48, wherein the first engagement structure engages a fluid outlet end of one of the first syringe and the second syringe and the second engagement structure engages a fluid outlet end of the other of the first syringe and the second syringe in a manner to prevent proximal and distal movement of the first syringe and the second syringe during a fluid filling and fluid injection procedure.

Clause 51. The syringe system of any of clauses 35 to 50, wherein the body structure further comprises at least one spike in fluid communication with at least one of the first syringe and the second syringe for connection of at least one bulk fluid source with the at least one of the first syringe and the second syringe.

Clause 52. The syringe system of clause 51, wherein the at least one spike is provided in fluid communication with at least one of the first syringe and the second syringe via at least one of tubing or a manifold associated with the frame.

Clause 53. The syringe system of any of clauses 35 to 52, wherein the first engagement member and the second engagement member are configured as a first pressure jacket cap and a second pressure jacket cap, respectively, each comprising a frusto-conical structure that engages and surrounds a frusto-conical distal end of each of the first syringe and the second syringe.

Clause 54. The syringe system of any of clauses 35 to 52, wherein the first engagement member comprises a first syringe cap and the second engagement member comprises a second syringe cap, and wherein the first syringe cap and a first portion of the body structure is connected to the second syringe cap and a second portion of the body structure by an engagement mechanism provided between the first portion of the body structure and the second portion of the body structure.

Clause 55. The syringe system of clause 54, wherein the engagement mechanism permanently connects the first portion of the body structure and the second portion of the body structure or removably connects the first portion of the body structure and the second portion of the body structure.

Clause 56. The syringe system of clause 54 or 55, wherein the first portion of the body structure and the second portion of the body structure are color coded according to the type of fluid to be used in the first and second syringes.

Clause 57. The syringe system of any of clauses 35 to 56, wherein the first engagement member, the second engagement member, the body structure, the first syringe, and the second syringe are integrally formed.

Clause 58. The syringe system of any of clauses 35 to 57, wherein the body structure comprises at least one identification component provided thereon configured to be read by the powered injector system to provide information to the powered injector system.

Clause 59. The syringe system of clause 58, wherein the information comprises at least one of information related to the first syringe and the second syringe, information related to an injection procedure, information related to the patient, or any combination thereof.

Clause 60. A syringe for a powered injector system, the syringe comprising: a proximal end, a distal end, and a cylindrical sidewall between the proximal end and the distal end defining an interior volume for retaining a medical fluid therein; a discharge nozzle at the distal end; a piston engagement feature located on one of plunger slidably associated with the syringe and a proximal end wall of the syringe, the piston engagement feature configured for releasably engaging a piston of the power injector system; and a frame positioned at the distal end of the syringe, wherein the frame is configured to index the syringe with the power injector system such that the syringe is held in a proper vertical position within the powered injector system for conducting a fluid injection procedure.

Clause 61. The syringe of clause 60, wherein the frame comprises a support structure extending therefrom.

Clause 62. The syringe of clause 61, wherein the support structure is configured to support and manage at least one of: tubing used to fill the syringe; spikes used to connect the syringe to a bulk fluid source; a patient tubing set; a priming tube; and any combination thereof.

Clause 63. The syringe of clause 61 or 62, wherein the support structure comprises an oval portion configured to manage at least one of at least one tubing used to fill the syringe and at least one spike used to connect the syringe to a bulk fluid source.

Clause 64. The syringe of clause 63, wherein the oval portion comprises clips on a top surface thereof configured to secure at least a portion of the patient tubing set to the oval portion.

Clause 65. The syringe system of any of clauses 61 to 64, wherein the support structure is molded as a first half and second half with the first half and the second half are connected to the frame via a hinge mechanism such that the first half and the second half can be connected together to form the support structure by closing the hinge mechanism.

Clause 66. The syringe of any of clauses 60 to 65, wherein the frame comprises at least one spike in fluid communication with the syringe for fluidly connecting of at least one bulk fluid source with the syringe.

Clause 67. The syringe of clause 66, wherein the at least one spike is provided in fluid communication with the syringe via at least one of tubing or a manifold associated with the frame.

Clause 68. The syringe of clause 60, wherein the frame comprises an engagement mechanism for connecting the frame to a second frame of a second syringe, wherein the connected frame and second frame are configured to index the syringe and the second syringe with the power injector system such that the syringe and second syringe are held in a proper vertical position within the powered injector system for conducting a fluid injection procedure.

Clause 69. The syringe of clause 68, wherein the engagement mechanism permanently connects the frame to the second frame of the second syringe or removably connects the frame to the second frame of the second syringe.

Clause 70. The syringe of any of clauses 60 to 69, wherein the frame and the syringe are integrally formed.

Clause 71. The syringe of any of clauses 60 to 70, wherein the frame comprises at least one identification component provided thereon configured to be read by the powered injector system to provide information to the powered injector system.

Clause 72. The syringe of clause 71, wherein the information comprises at least one of information related to the syringe, information related to an injection procedure, information related to the patient, or any combination thereof.

These and other features and characteristics of the device of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the device of the present disclosure. As used in the specification and the claims, the singular form of “a”, “an”, and “the” include plural referents unless the context clearly dictates otherwise.

### BRIEF DESCRIPTION OF THE DRAWINGS

FIG. 1 is a perspective view of a fluid delivery system with pressure jackets for use with a rolling diaphragm syringe according to an example of the present disclosure;

FIGS. 2A and 2B are side cross-sectional views of an unrolled and rolled rolling diaphragm syringe, respectively, configured for use with the syringe frames according to various embodiments and with the fluid delivery system of FIG. 1;

FIGS. 3A and 3B illustrate embodiments of a sliding syringe cap having separate filling and delivery paths for use with the syringe of FIGS. 2A and 2B;

FIG. 4 is a perspective view of a frame for engaging a pair of syringes in accordance with an aspect of the present disclosure;

FIG. 5 is a perspective view of a frame for engaging a pair of syringes in accordance with another aspect of the present disclosure;

FIG. 6A is a front view of a frame having a support structure for securing a valve assembly in accordance with still further aspects of the present disclosure; FIG. 6B is a top view of the frame of FIG. 6A in an open position; and FIG. 6C is a top view of the frame of FIG. 6A in a closed position;

FIG. 7 is a front view of a frame having an alternative example of a support structure in accordance with the present disclosure;

FIG. 8 is a perspective view of the frame of FIG. 4 having a clamping support structure in accordance with the present disclosure;

FIG. 9 is a perspective view of a frame for engaging a pair of syringes in accordance with another aspect of the present disclosure with the frame being in an open configuration;

FIG. 10 is a front view of the frame of FIG. 9 engaged with a pair of syringes and provided in a fluid injector in accordance with the present disclosure;

FIG. 11A is a perspective view of an alternative example of a frame in an open configuration in accordance with the present disclosure;

FIG. 11B is a perspective view of the frame of FIG. 11A engaged with a pair of syringes in accordance with another aspect of the present disclosure;

FIGS. 12A and 12B are perspective views of a frame for engaging a pair of syringes in accordance with another aspect of the present disclosure with the frame attached and detached from a pair of syringes;

FIG. 13A is a perspective view of a frame for engaging a pair of syringes and a bulk fluid source in accordance with another aspect of the present disclosure;

FIG. 13B is a perspective view of the frame of FIG. 13A illustrating a first step of a syringe filling procedure;

FIG. 13C is a perspective view of the frame of FIG. 13A illustrating a second step of a syringe filling procedure;

FIG. 14A is a perspective view of a frame for engaging a pair of syringes and a bulk fluid source in accordance with another aspect of the present disclosure;

FIG. 14B is a perspective view of the frame of FIG. 14A illustrating a first step of a syringe filling procedure;

FIG. 14C is a rear perspective view of the frame of FIG. 14B illustrating a second step of a syringe filling procedure;

FIG. 15A is a perspective view of a frame for engaging a pair of syringes and a bulk fluid source in accordance with another aspect of the present disclosure;

FIG. 15B is a perspective view of the frame of FIG. 15A illustrating a first step of a syringe filling procedure;

FIG. 15C is a rear perspective view of the frame of FIG. 15A illustrating a second step of a syringe filling procedure;

FIG. 16A is a perspective view of a frame for engaging a pair of syringes and a bulk fluid source in accordance with another aspect of the present disclosure;

FIG. 16B is a second rear perspective view of the frame of FIG. 16A;

FIG. 16C is a rear perspective view of the frame of FIG. 16A illustrating a syringe filling procedure;

FIG. 17A is a perspective view of a syringe cap for use with a single syringe, and FIG. 17B is a frame comprising a pair of the syringe caps of FIG. 17A in accordance with the present disclosure;

FIG. 18 is a perspective view of a frame for use with a single syringe in accordance with an aspect of the present disclosure;

FIG. 19 is a perspective view of a frame for use with a single syringe in accordance with another aspect of the present disclosure;

FIG. 20 is a perspective view of a frame for use with a single syringe in accordance with still another aspect of the present disclosure; and

FIG. 21 is a perspective view of a frame for engaging a pair of syringes in accordance with an aspect of the present disclosure.

### DETAILED DESCRIPTION

For purposes of the description hereinafter, the terms “upper”, “lower”, “right”, “left”, “vertical”, “horizontal”, “top”, “bottom”, “lateral”, “longitudinal”, and derivatives thereof shall relate to the components as they are oriented in the drawing figures. When used in relation to a syringe and/or a pressure jacket, the term “proximal” refers to a portion of a syringe and/or a pressure jacket nearest to an injector when a syringe and/or a pressure jacket is oriented for connecting to an injector. The term “distal” refers to a portion of a syringe and/or a pressure jacket farthest away from an injector when a syringe and/or a pressure jacket is oriented for connecting to an injector. The term “radial” refers to a direction in a cross-sectional plane normal to a longitudinal axis of a syringe and/or a pressure jacket extending between proximal and distal ends. The term “circumferential” refers to a direction around an inner or outer surface of a sidewall of a syringe and/or a pressure jacket. The term “axial” refers to a direction along a longitudinal axis of a syringe and/or a pressure jacket extending between the proximal and distal ends. The term “flexible”, when used in connection with a syringe, means that at least a portion of a syringe, such as a sidewall of a syringe, is capable of bending or being bent to change a direction in which it extends. The terms “roll over”, “rolling over”, and “rolls upon itself” refer to an ability of a first portion of a syringe, such as a proximal portion of a sidewall of a syringe, to bend approximately 180° relative to a second portion of a syringe, such as a distal portion of a sidewall of a syringe, when urged by a piston of a fluid injector. The term “closed” when used to refer to a fluid delivery component means that the system is not in fluid connection with an outlet, for example where fluid flow is stopped by a cap, a closure member, or a valve, such as a stopcock, high crack pressure valve, pinch valve, and the like.

Unless otherwise indicated, all ranges or ratios disclosed herein are to be understood to encompass any and all subranges or sub-ratios subsumed therein. For example, a stated range or ratio of “1 to 10” should be considered to include any and all subranges between (and inclusive of) the minimum value of 1 and the maximum value of 10; that is, all subranges or sub-ratios beginning with a minimum value of 1 or more and ending with a maximum value of 10 or less, such as but not limited to, 1 to 6.1, 3.5 to 7.8, and 5.5 to 10.

All documents, such as but not limited to issued patents and patent applications, referred to herein, and unless otherwise indicated, are to be considered to be “incorporated by reference” in their entirety.

Referring to the drawings in which like reference characters refer to like parts throughout the several views thereof, the present disclosure is generally directed to syringe collar and frame elements for managing the syringe arrangements and patient tubing and filling components for ease of use in the preparation of a fluid injector for a fluid injection procedure. These syringe collars and frames are considered to be a part of the packaging design for the rolling diaphragm syringe dual kit or rolling diaphragm single syringe kit. In general, the embodiments described herein were created at least in part for the purposes of improving workflow when engaging the syringes with the fluid injector; managing the patient tubing and filling components; and/or also providing a reference (datum) for the location of the syringes in the injection system. Accordingly, the basic purposes of the syringe collars and frames according to various embodiments described herein include: 1) indexing the distal end of the syringes within the injector so that they are held in the proper orientation and distance for insertion into the injector and engaging with one or more pistons of the injector; 2) managing the tubing (fill and patient) and spikes used for an injection procedure by providing a scaffold for storage and management of the tubing and spikes; 3) providing a handle for the user to quickly and easily install and remove the syringe(s) within the injector; and 4) improving workflow by requiring fewer steps for setup of the injection procedure.

In many medical diagnostic and therapeutic procedures, a medical practitioner, such as a physician, injects a patient with one or more medical fluids. In recent years, a number of injector-actuated syringes and powered fluid injectors for pressurized injection of medical fluids, such as an imaging contrast solution (often referred to simply as “contrast”), a flushing agent, such as saline, and other medical fluids, have been developed for use in procedures such as cardiovascular angiography (CV), computed tomography (CT), ultrasound, magnetic resonance imaging (MRI), positron emission tomography (PET), and other imaging procedures. In general, these fluid injectors are designed to deliver a preset amount of fluid at a preset pressure and/or flow rate. According to various embodiments, the fluid injector may be a dual head fluid injector having two syringes, including a contrast syringe injecting with an imaging contrast medium and a saline syringe for injecting a medical flushing agent, such as saline.

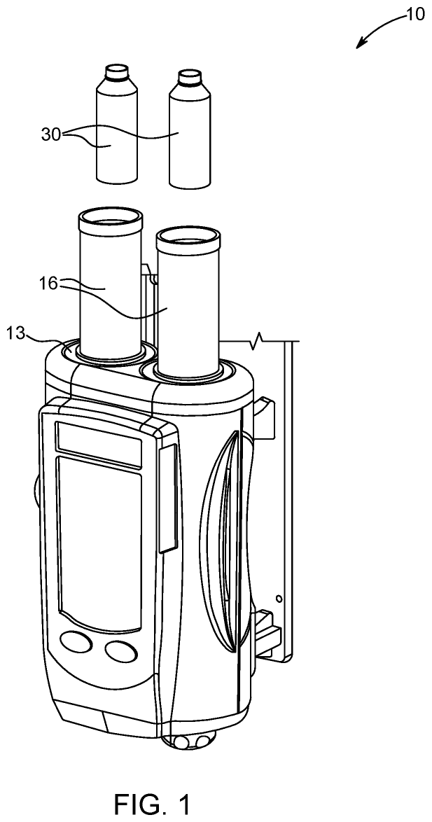

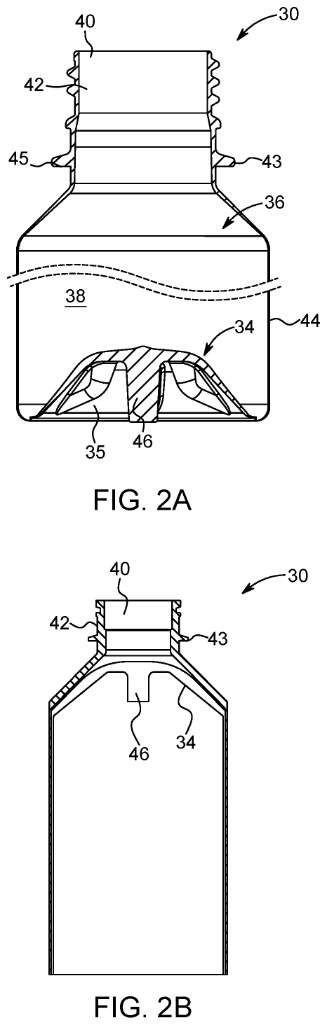

Typically, fluid injectors have at least one drive member, such as pistons, that connects to the syringe, for example via connection with a plunger or an engagement feature on and end wall of the syringe. Conventional syringe generally includes a rigid barrel with the syringe plunger being slidably disposed within the barrel. In other embodiments, the syringe may include a rolling diaphragm barrel configuration having a flexible sidewall, where the proximal end of the syringe body releasably interacts with the at least one drive member, as generally described, for example in U.S. Patent Application Publication Nos. 2017/0035974 and 2018/0161496, the disclosures of which are incorporated herein by this reference. The drive members drive the plungers or the rolling diaphragm/proximal end wall in a proximal and/or distal direction relative to a longitudinal axis of the barrel to draw fluid into the syringe barrel or deliver the fluid from the syringe barrel, respectively. Non-limiting examples of a fluid injector and rolling diaphragm syringe suitable for use with the syringe collars of the present disclosure is described in FIGS. 1, 2A and 2B.

With reference to FIG. 1, the fluid injector 10, such as an automated or powered fluid injector, may include at least one syringe including a compressible sidewall, for example, a rolling diaphragm 30, configured to be filled with a fluid and to administer the fluid to a patient during a fluid injection procedure. The fluid injector may be configured to receive the at least one syringe within at least one pressure jacket 16. The pressure jacket 16 is typically a reusable component configured to be releasably engaged with a fluid injector port 13, while the syringe 30 is typically a single-use component configured to be discarded after an injection procedure. In other embodiments, the syringe 30 may be a multi-use component for use in several injection procedures before disposal. The fluid injector 10 may have at least one bulk fluid source for filling the at least one syringe with a fluid, for example through the fluid inlet path. The bulk fluid source may be a first bulk fluid source (not shown) containing a first medical fluid, such as an imaging contrast medium, and a second bulk fluid source (not shown) containing a second medical fluid, such as saline or Ringer's lactate, for filling a first and second syringe with first or second fluid contained in the first and second bulk fluid sources, respectively. At least one fluid path set (not shown) may be fluidly connected with a distal discharge end of the at least one syringe for delivering fluid from the syringe through tubing connected to a catheter, needle, or other fluid delivery connection (not shown) inserted into a patient at a vascular access site. Fluid flow into and from the syringe may be regulated by a fluid control module (not shown) associated with the fluid injector and/or by proximal or distal movement of the syringe causing slidable movement of a sliding syringe cap (discussed herein and in PCT International Application Publication No. WO2019/055497, the disclosure of which is incorporated herein by reference) associated with a cap or other controllable valve provided at or near the distal end of the syringe. The fluid control module may operate various pistons and/or flow regulating structures to regulate the delivery of the medical fluid, such as saline solution and contrast and dual flow mixtures thereof, to the patient based on user selected injection parameters, such as injection flow rate, duration, total injection volume, and/or ratio of contrast media and saline. Examples of suitable front-loading fluid injectors that may be used or modified for use with the herein-described system, including at least one pressure jacket and syringe, are disclosed in International Patent Application Publication Nos. WO2015/164783 and WO2016/172467, and PCT Application No. PCT/US2019/050293, the disclosures of each of which are incorporated herein by reference.

With continued reference to FIG. 1, the injector 10 may be a multi-syringe injector having two, three, or more syringes 30, wherein the several syringes 30 may be oriented in a side-by-side or other relationship and may be separately actuated by respective drive members or pistons associated with the injector 10. In examples with two or more syringes, for example, arranged in a side-by-side or other relationship and filled with two different fluids, the injector 10 may be configured to deliver fluid from one or both of the syringes 30, sequentially or concurrently. According to one embodiment, the fluid injector 10 may be a dual head injector having two syringes 30. The first syringe 30 may be for delivering a contrast media or other medical fluid and a second syringe 30 may be for delivering saline or other medically approved flushing agent to flush the contrast media to the patient. According to various embodiments, the fluid injector 10 may be configured to deliver the contrast and saline separately (e.g., delivering a specific volume saline over a specific time followed by delivering a specific volume of contrast over a specific time, followed by a second volume of saline over a specified time to flush the contrast media from the tubing into the patient). According to various embodiments, the fluid injector 10 may be configured to deliver the contrast and saline separately or as a mixture (e.g., delivering a specific volume saline over a specific time followed by delivering a specific volume of contrast or a specified ratio of contrast and saline (i.e., in a “dual flow” process) over a specific time, followed by a second volume of saline over a specified time to flush the contrast media from the tubing into the patient). A technician may program a specific injection protocol into the injector or use a pre-written protocol to deliver the desired volumes of saline, contrast, specific ratios of contrast and saline mixtures, etc., at desired flow rates, times, and volumes for each solution. The fluid injector 10 may have at least one bulk fluid source (not shown) for filling the syringes 30 with fluid and in certain embodiments, the fluid injector 10 may have a plurality of bulk fluid source, one for each of the plurality of syringes 30, for filling each of the plurality of syringes 30 with the desired fluid.

A fluid path set (not shown) may be in fluid communication with each syringe 30 to place each syringe in fluid communication with a catheter for delivering the fluid from each syringe to a catheter (not shown) inserted into a patient at a vascular access site. In certain embodiments, fluid flow from the one or more syringes 30 may be regulated by a fluid control module (not shown) that operates various drive members, pistons, valves, stopcocks, syringe engagement/disengagement mechanisms, and various flow regulating structures to regulate the delivery of the saline solution and contrast to the patient based on user selected injection parameters, such as injection flow rate, duration, total injection volume, and ratio of fluids from the syringes 30, including specific ratios of each fluid in a dual flow injection protocol.

In some examples, such as shown in FIG. 1, the injector 10 may be configured for receiving and retaining the pressure jacket 16 within each syringe port 13 of the injector 10. While FIG. 1 illustrates a fluid injector 10 with two syringe ports 13, each having a corresponding pressure jacket 16, other examples of the fluid injector 10 may include more than two syringe ports 13 with an optional corresponding number of pressure jackets 16. In embodiments comprising pressure jackets, each pressure jacket 16 may be configured to receive a syringe, such as a syringe for a cardiovascular angiographic (CV) procedure, CT procedure and in various embodiments a rolling diaphragm syringe 30 (suitable examples of which are described in described in International Patent Application Publication Nos. WO2018/075386; WO2016/172467; and WO2015/164783). A fluid path set (not shown) may be fluidly connected with a discharge end or port connected with each rolling diaphragm syringe 30 for delivering fluid from the syringes 30 through tubing connected to a fluid delivery connection (not shown) inserted into a patient at a vascular access site. According to various embodiments, the syringe 30 may be a pre-filled syringe, e.g., a pre-filled rolling diaphragm, i.e., the syringe may be prefilled with a medical fluid when provided by the manufacturer.

With reference to FIGS. 2A and 2B, an exemplary rolling diaphragm syringe 30 includes a hollow body 36 defining an interior volume 38. The body 36 has a forward or distal end 40, a rearward or proximal end 35, and a flexible sidewall 44 extending therebetween. The proximal end 35 may be configured to act with a piston to pressurize the syringe interior to expel fluid therefrom or draw fluid therein. The sidewall 44 of the rolling diaphragm syringe 30 defines a soft, pliable or flexible, yet self-supporting body that is configured to roll upon itself, as a “rolling diaphragm”, under the action of the drive member or piston of the fluid injector 10. A drive member/piston (not shown) of the injector may be configured to releasably engage a drive member engagement portion 46 at the proximal end 35 of the rolling diaphragm syringe 30 (examples of which are described in International Patent Application Publication No. WO2018/075386). In operation, the sidewall 44 is configured to roll such that its outer surface is folded and inverted in a radially inward direction as the drive member/piston moves the proximal end 35 in a distal direction and unrolled and unfolded in the opposite manner in a radially outward direction as the drive member/piston retract the proximal end 35 in a proximal direction.

With continued reference to FIGS. 2A and 2B, the proximal portion of the sidewall 44 connects to a closed end wall 34, and a distal portion of the sidewall 44 defines a discharge neck 42. The closed end wall 34 may have a concave shape to facilitate the initiation of the inversion or rolling of the sidewall 44, enhance mechanical strength of the closed end wall 34, and/or to provide a receiving pocket to receive a distal end of the drive member/piston of the injector 10. The drive member engagement portion 46 is configured for releasably interacting with a corresponding engagement mechanism on the drive member/piston, for example as the drive member/piston is retracted. In use, the proximal end 35 of the syringe 30 is configured for insertion into the throughbore of a pressure jacket 16 such that the sidewall 44 is surrounded by the interior surface of the pressure jacket 16. At least a portion of the distal end 40 of the syringe 30 may extend from a distal end of the pressure jacket 16. The distal end 40 may have a frusto-conical shape that gradually narrows from the sidewall 44 to the discharge neck 42. At least one retention flange 43 may extend around the circumferential surface of the discharge neck 42. The retention flange 43 may extend around the entire circumferential surface of the discharge neck 42 or a portion of the circumferential surface of the discharge neck 42. The retention flange 43 may have a proximal surface 45 for interacting with a corresponding feature of the syringe frames described herein to limit a distance that the syringe slides into the pressure jacket 18 when a user engages the at least one syringe and syringe frame with the fluid injector.

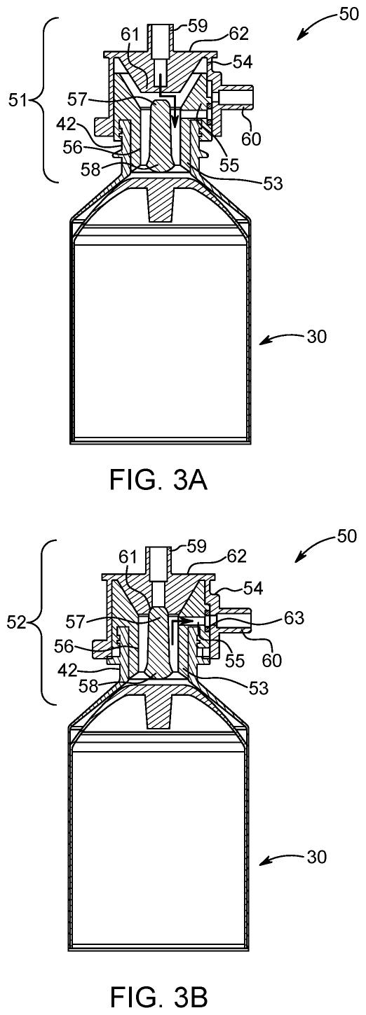

With reference to FIGS. 3A and 3B, in some embodiments, the syringe 30 may include an active sliding syringe cap 50 that is configured to slide between a first filling position 51 where the syringe 30 is in fluid communication with a bulk fluid container for filling the syringe 30 and a second delivery position 52 where the syringe 30 is in fluid communication with a fluid path for delivery of fluid to a patient. In certain embodiments, the sliding syringe cap 50 may have a third closed position where the interior volume 38 of the syringe 30 is fluidly isolated. Referring first to FIG. 3A, the active sliding syringe cap 50 is shown in the first filling position 51. Active sliding syringe cap 50 is shown in fluid tight connection with a distal discharge neck 42 of the rolling diaphragm syringe 30. The sliding syringe cap 50 comprises an inner cap assembly 53 in slidable engagement with an outer cap assembly 54. The inner cap assembly 53 includes an inner delivery fluid path 55, a syringe fluid path 56, a pin 57, and a flow controller 58. The outer cap assembly 54 includes a fluid inlet path 59, a fluid outlet path 60, a pin abutment feature 61, and a flat distal surface 62 to interact with a retaining surface of a holding bracket of the injector 10 or with a portion of the frame as discussed herein. Examples of embodiments of the active sliding syringe cap 50 are described in PCT International Application Publication No. WO2019/055497.

With continued reference to FIG. 3A, in the first filling position 51, fluid communication is established between the fluid inlet path 59 and the interior of the syringe 30 during a filling procedure. A fluid container is attached by a fluid line to the fluid inlet path 59 and fluid may flow into syringe 30. In the first filling position, fluid communication between the inner delivery fluid path 55 and the fluid outlet path 60 is prevented. Fluid flows past flow controller 58, which forces the fluid to flow down the inner sidewall of syringe 30.

FIG. 3B illustrates the active cap 50 in a second delivery position 52. In the second delivery position 52, the outer cap assembly 54 has slid proximally relative to the inner cap assembly 53 due to distal movement of the piston and end wall 34 of the syringe 30 to allow fluid communication between the inner delivery fluid path 55 and syringe 30 and fluid outlet path 60 via syringe fluid path 56. Fluid communication between the fluid inlet path 59 and syringe fluid path 56 is prevented by abutment of the pin 57 with the pin abutment feature 61.

To improve work flow for an operator of an injection protocol, the present disclosure provides a series of syringe frames that engage and hold one or more syringes, such as rolling diaphragm syringe 30, in a configuration for easy packaging, manipulation, and insertion/removal into the pressure jackets 16 associated with a fluid injector 10. In addition, embodiments of the frame concepts may include bulk fluid container, tubing, and/or spike management features that further improve workflow. While the present disclosure describes the frames and tubing/spike management solutions as shown with a rolling diaphragm-type syringe, the solutions may also be applied to conventional front loading syringes and injectors such as described in U.S. Patent Nos. 5,383,858; 7,553,294; 7,666,169; 9,173,995; 9,199,033; and 9,474,857; and in International Patent Application Publication Nos. WO2016/191485 and WO2016/112163, the disclosures of each of which are incorporated by reference in their entirety. As discussed herein, embodiments of the frames described herein were created for improving workflow by capturing the syringes and managing the patient tubing and filling components and also providing a reference (datum) for the location of the syringes in the injection system. For example, the frames may orient and locate the at least one syringe relative to each other, the distal end of the pressure jackets and/or the engagement features on the pistons so that the syringe/frame assembly may be easily inserted. Further, the frame may determine the distance that the one or more syringes are inserted into the pressure jacket for optimal engagement with the piston without the syringe falling downward into the pressure jacket. Further, the frame may allow indexing of the front retaining cap relative to the injector, thus allowing for ready insertion/ removal of the assembly and so injector knows where the proximal end of the at least one syringe should be. Accordingly, various purposes of the syringe collars and frames according to various embodiments described herein include: 1) indexes the syringes within the injector so that they are held in the proper orientation and distance for insertion; 2) managing tubing (fill and patient) and spikes used in an injection procedure by providing a scaffold for storage and management of tubing and spikes; 3) providing a handle for the user to quickly and easily install and remove the syringe(s) within the injector without touching and potentially contaminating a surface of the syringe; and 4) improving workflow by requiring fewer steps to setup the injection procedure.

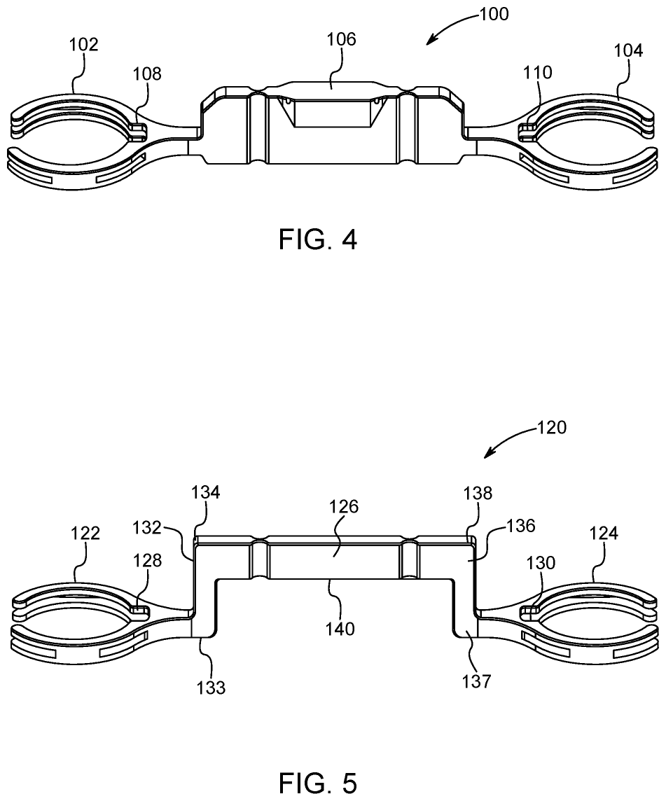

With reference to FIG. 4, one example of a frame 100 comprises a first engagement member 102 configured to secure at least a portion of a first syringe 30 and a second engagement member 104 configured to secure at least a portion of the second syringe 30. The first engagement member 102 and the second engagement member 104 are generally C-shaped and may be configured to engage the retention flange 43 or other feature provided on the distal end 40 of each of the first and second syringes 30 or on a syringe cap associated with the first and second syringes 30. In addition, the first and second engagement members 102, 104 may each include a notch 108, 110 or other feature to accommodate the cap 50 that may be positioned over the distal end 40 of the first and second syringes 30. While first and second engagement members 102, 104 are illustrated as C-shaped, other configurations providing suitable interaction with the at least one syringe 30 are contemplated.

The frame 100 further includes a body structure 106 connected to the first engagement member 102 and the second engagement member 104. The body structure 106 is sized and shaped to index the first and second syringes 30 with the fluid injector 10 such that the first and second syringes 30 are held in an orientation and distance relative to each other for insertion into the fluid injector 10. Further, a bottom surface of the body structure 106 may interact with a corresponding surface of the fluid injector 10 to center and limit the depth of insertion and centering of the proximal end of the first and second syringes 30 within the corresponding pressure jackets. The body structure 106 may also provide a surface for at least one identification component to be provided thereon, where the at least one identification component may be configured to be read by a processor or optical scanner associated with the powered injector system and provide information to the injector system, such as information related to the at least two fluid containers, information related to an injection procedure, information related to the patient, or any combination thereof. In certain embodiments, the at least one identification component may be in the form of a bar code, QR-code, or other identifying indicia.

Accordingly, frame 100 is to be used for positioning the syringes 30 in the injector 10 while providing a handle for the user to easily grasp both syringes 30 to place into the injector 10. The first engagement member 102, the second engagement member 104, and/or the body structure 106 may include one or more feature that interacts with the injector to create audible and/or tactile feedback to indicate to the user that the syringes 30 are correctly positioned within the injector 10.

The frame 100 may be made of any suitable medical-grade plastic or polymeric material. In certain examples, the polymeric material is a clear or substantially translucent plastic material such as when used in an injector described in U.S. Patent Application Publication No. 2017/0056603, the disclosure of which is incorporated by reference, to help illuminate the light source provided by such an injector. In other embodiments, the polymeric material may have any other color for cost, manufacturing, and/or aesthetic purposes. Any suitable alternative material may also be used to manufacture the frame 100.

In another embodiment as shown in FIG. 23, the frame 100A comprises a first engagement member 102A configured to secure at least a portion of a first syringe 30 and a second engagement member 104A configured to secure at least a portion of the second syringe 30. The frame 100A further includes a body structure 106A connected to the first and second engagement members 102A, 104A. The body structure 106A is sized and shaped to index the first and second syringes 30 with the fluid injector 10 such that the first and second syringes 30 are held in an orientation and distance relative to each other for insertion into the fluid injector 10 In addition, the body structure 106A includes a feature 107 having at least one notch 109 for receiving and managing tubing and/or spikes. The body structure 106A is also provided with a pair of prongs 111 for engaging with a feature of the injector for orienting, locating, and/or locking the frame in place. At least a portion of body structure 106A may be configured as flat, planar structures designed to fit within a corresponding slot at a distal end of the fluid injector to further orient the syringes relative to the fluid injector.

With reference to FIG. 5, another embodiment of a frame 120 according to the present disclosure comprises a first engagement member 122 configured to secure at least a portion of a first syringe 30 and a second engagement member 124 configured to secure at least a portion of a second syringe 30. Similar to the example shown in FIG. 4, the first engagement member 122 and the second engagement member 124 are generally C-shaped and may be configured to engage the retention flange 43 or other feature provided on the distal end 40 of each of the first and second syringes 30 or on a syringe cap associated with the first and second syringes 30. In addition, the first and second engagement members 122 may each include a notch 128, 130 or other feature to accommodate the cap 50 that may be positioned over the distal end 40 of the first and second syringes 30. While the first and second engagement members 122, 124 are illustrated a C-shaped, other configurations that provide suitable interaction and connection with the at least one syringe 30 are contemplated.

As in the embodiment illustrated in FIGS. 4, the frame 120 includes a body structure 126 connected to the first engagement member 122 and the second engagement member 124 is illustrated in FIG. 5. The body structure 126 is sized and shaped to index the first and second syringes 30 with the fluid injector 10 such that the first and second syringes 30 are held in an orientation and distance relative to each other for insertion into the fluid injector 10. In addition, the body structure 126 comprises a first leg 132 having a first end 133 extending vertically from the first engagement member 122 and a second end 134, a second leg 136 having a first end 137 extending vertically from the second engagement member 124 and a second end 138, and a body member 140 connected between the second end 134 of the first leg 132 and the second end 138 of the second leg 136. Further, a bottom surface of the body member 140 may interact with a corresponding surface of the fluid injector 10 to center and limit the depth of insertion of the proximal ends of the first and second syringes 30 within the corresponding pressure jackets while indexing the position of the top of the syringe relative to the injector. The body structure 126 may also provide a surface for at least one identification component to be provided thereon. The lengths of the first leg 132 and the second leg 136 are also selected to center and provide a proper vertical indexing of the top and insertion depth of the end wall of the first and second syringes 30 when positioned within the pressure jackets 16 of the fluid injector 10.

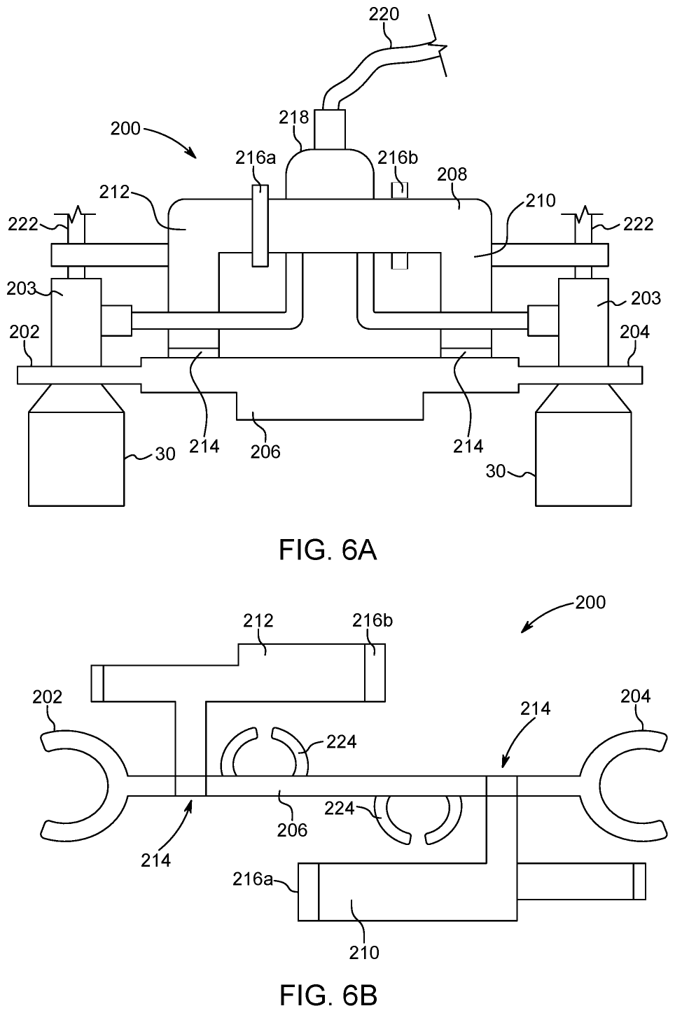

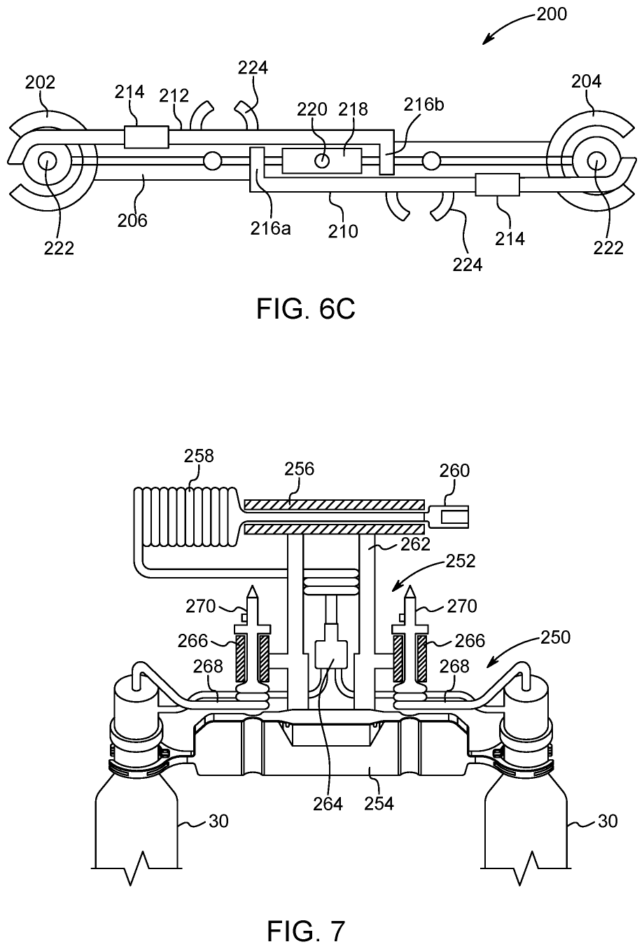

The frames of the present disclosure may further include at least one support structure for supporting and managing the tubing sets (fill sets and patient sets) and spikes used for an injection procedure. An example of such a frame is shown in FIGS. 6A, 6B, and 6C. Specifically, the frame 200 comprises a first engagement member 202 configured to secure at least a portion of a first syringe 30 and/or a first syringe cap 203, a second engagement member 204 configured to secure at least a portion of a second syringe 30 and/or a second syringe cap 203, and a body structure 206 connected to the first engagement member 202 and the second engagement member 204. Syringe cap 203 may be an active syringe capor a passive syringe cap, as described herein. As with the previously described embodiments, the body structure 206 is sized and shaped to index the first and second syringes 30 relative to the fluid injector 10 such that the first and second syringes 30 are held in an orientation and distance relative to each other for proper insertion into fluid injector 10.

The frame 200 further comprises a support structure 208 extending distally from the body structure 206. The support structure 208 comprises a first support structure portion 210 and a second support structure portion 212. The first support structure portion 210 and the second support structure portion 212 are each connected to the body structure 206 via a hinge mechanism 214 such that the support structure 208 may be provided in and hingedly move between a first opened position (see FIG. 6B) and a second, closed supporting position (see FIGS. 6A and 6C). The first support portion 210 and second support portion 212 further include at least one fastening mechanism 216a, 216b thereon for securing first support portion 210 and second support portion 212 in the closed position and providing a suitable connection for retaining one or more tubing systems and/or valve assembly 218. Fastening mechanisms 216a, 216b may be configured as any suitable closure, fastener, or snap.

The support structure 208 may be configured to secure a valve assembly 218 in fluid communication with outlets associated with a first cap and a second cap of the first and second syringes 30, respectively. The valve assembly 218 is provided to selectively deliver fluid to a patient fluid tubing set 220 from one of the syringes 30 while preventing fluid from being delivered to the patient fluid tubing set 220 from the other syringe 30 or for selectively delivering a dual flow ratio of a first fluid and a second fluid from a first and second syringe, respectively. Suitable examples of valve assembly 218 are described in detail in PCT International Application Publication No. WO2018/075390, the disclosure of which is incorporated by this reference. Support structure 208 may also include structures for supporting and managing tubing 222 used to fill or deliver fluid from the syringes 30. In some examples, the body structure 206 may be provided with clips 224 for securing spikes, prime tubes, fill tubes and the like thereto. The clips 224 may be provided on one or both sides of the body structure 206.

With reference to FIG. 7, in yet another embodiment, the frame 250 may further comprise a support structure 252 formed as scaffolding extending distally from the body structure 254 thereof. The body structure 254 is sized and shaped to index the first and second syringes 30 with the fluid injector 10 such that the first and second syringes 30 are held in an orientation and distance relative to each other for insertion into the fluid injector 10. The scaffolding of the support structure 252 includes various attachment portions configured to secure various elements required for a fluid injection procedure such as, but not limited to, an attachment portion 256 for patient tubing 258 and/or a prime tube 260, an attachment portion 262 for a valve assembly 264 in fluid communication with outlets of the first and second syringes 30 and patient tubing 258, and attachment portions 266 for fill tubing 268 and spikes 270 used to fill the first and second syringes 30 from one or more bulk fluid sources (not shown). In other embodiments, the prime tube 260 may be molded as part of the support structure 252.

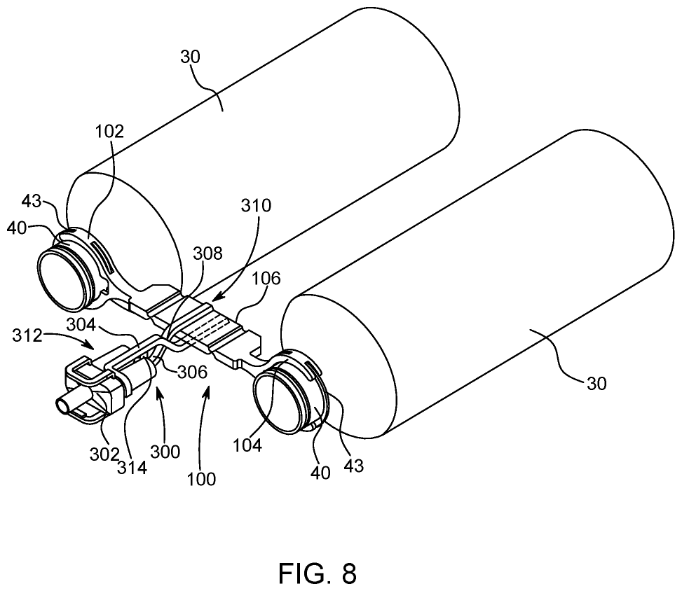

While the support structure of the frames may be integrally formed with other frame structures as shown in FIGS. 6A, 6B, 6C, and 7, in some examples, the support structure may be provided as a separate element configured to be attached, adhered to, or otherwise connected to the body structure of the frame. For example, a support structure 300 designed to secure a valve assembly 302 to the frame 100 (see FIG. 4) is illustrated in FIG. 8. The support structure 300 comprises a first clamping element 304 and a second clamping element 306 designed to engage each other at a central pivot point 308 to create a first clamp 310 and a second clamp 312. The first clamp is designed to be secured to the body structure 106 of the frame 100 and the second clamp 312 is designed to secure the valve assembly 302 at a position between the first and second syringes 30. Similar to valve assembly 218 described hereinabove, the valve assembly 302 is provided in fluid communication with outlets of the first and second syringes 30 and is provided to deliver fluid to a patient fluid tubing set from one of the syringes 30 while preventing fluid from being delivered to the patient fluid tubing set from the other syringe 30 or for selectively delivering a dual flow ratio of a first fluid and a second fluid from a first and second syringe, respectively. Once the first clamp 310 and the second clamp 312 have been secured, a fastening mechanism 314 is provided to lock the support structure 300 in place. In one example, the fastening mechanism 314 comprises a first latch (not shown) extending from the first clamping element 304 designed to engage a second latch (not shown) extending from the second clamping element 306 where the first latch element and the second latch element engage to secure the first clamp 310 to the second clamp 312.

The various embodiments of the frame described herein may be used with syringes 30 including caps, such as, but not limited to caps as disclosed in International Patent Application Publication No. WO2019/055497 (FIGS. 3A and 3B). Such caps may include active valves that require distal and proximal movement of syringes 30 in order to change the state of the valve or passive valves where no such movement is required.

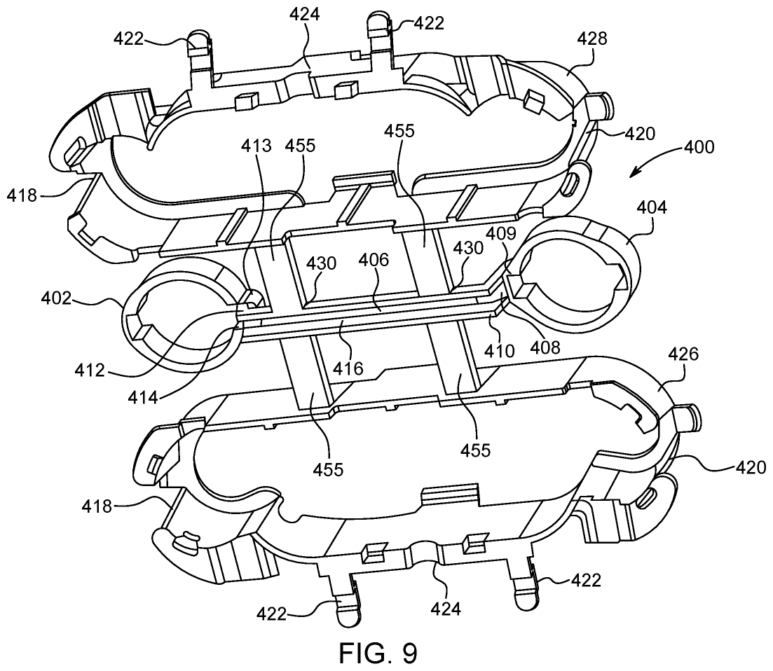

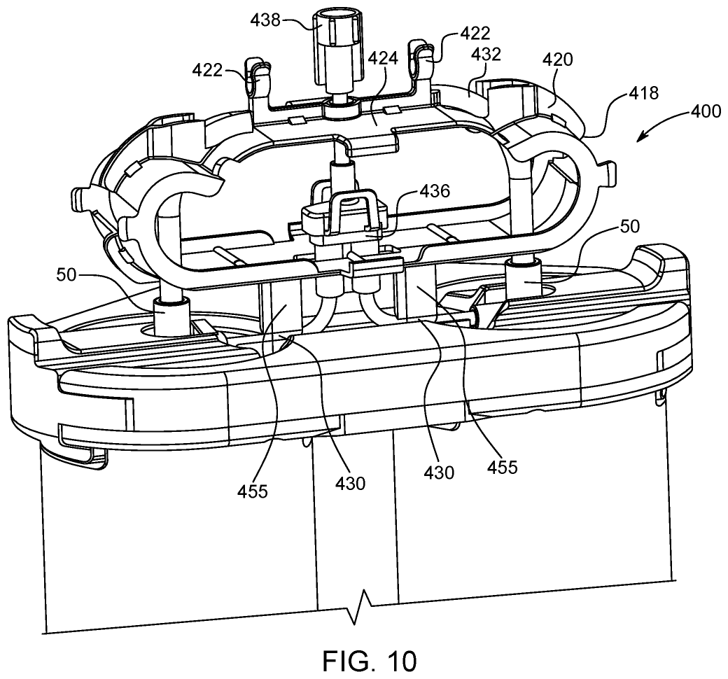

With reference to FIGS. 9 and 10, a frame 400 for use with a syringe 30 including a cap 50 having a valve is illustrated in a first open position (FIG. 9) and a second closed position (FIG. 10). The frame may be more readily molded (e.g., injection molded) in the first open position (FIG. 9) and may be adjusted to the second closed position when preparing the syringe assembly during manufacture. The frame 400 comprises a first engagement member 402 configured to secure at least a portion of a first syringe 30 and a second engagement member 404 configured to secure at least a portion of a second syringe 30. The first engagement member 402 and the second engagement member 404 are generally O-shaped and are configured to enclose and fit over the neck or finish of the syringes 30 before assembling the caps 50. The first and second engagement members 402, 404 may not be tightly constrained (locked into position) on the syringe finish, such that the engagement members can slide along an outer surface of the syringe neck 42 to allow for the syringe to move, for example when an active syringe valve is used. Alternatively, the first and second engagement members 402, 404 may be tightly constrained (locked into position) on the syringe finish, such that the engagement members cannot slide along an outer surface of the syringe neck 42 to limit movement of the syringe, for example when a passive syringe valve is used. However, while the first and second engagement members 402, 404 are illustrated as O-shaped, other configurations that provide suitable interaction and connection with the at least one syringe 30 are contemplated, as described herein.

The frame 400 further includes a body structure 406 connected to the first engagement member 402 and the second engagement member 404. As with other embodiments, the body structure 406 is sized and shaped to index the first and second syringes 30 with the fluid injector 10 such that the first and second syringes 30 are held in an orientation and distance relative to each other for insertion into the fluid injector 10 to a correct depth and centering within pressure jacket 16. In addition, the body structure 406 comprises a first leg 408 having a first end 409 extending vertically from the first engagement member 402 and a second end 410 attaching to body member 416, a second leg 412 having a first end 413 extending vertically from the second engagement member 404 and a second end 414 attached to body member 416, where body member 416 is connected between the second end 410 of the first leg 408 and the second end 414 of the second leg 410.

The frame 400 further includes a support structure 418 extending from the body structure 406. The support structure 418 includes an oval portion 420 for managing components various fluid paths and attachment elements used during a fluid injection procedure. In addition, the oval portion 420 may serve a handle for the user to easily grab the entire assembly and insert into the pressure jacket assembly of the injector. The oval portion 420 may further comprise clips 422 on a top surface 424 thereof configured to secure the patient tubing set as it encircles the outer surface of the oval portion 420.

As shown in FIG. 9, the support structure 418 is molded as a first half 426 and second half 428 with the first half 426 and the second half 428 connected the body structure 406 via a hinge mechanism 430 such that the first half 426 and the second half 428 can be connected together by closing the hinge structure 430 and connecting the first half 426 and the second half 428 together with a suitable fastening mechanism. The first half 426 and the second half 428 are connected to the body structure 406 by one or more vertical connection beams 455 that hold the support structure 418 distally away from the body structures 406. The fastening mechanism may be any suitable latch, clip, or snap. This design allows the packaging form factor to be small and compact while allowing ready injection molding without any undercuts within the mold form.

With reference to FIG. 10 and with continued reference to FIG. 9, the oval portion 420 is configured to secure and manage tubing 432 used to fill the syringes 30; one or more spikes (not shown) used to connect the syringes 30 to a bulk fluid source; and a valve assembly 436 that is provided in fluid communication with outlets of the syringes 30 and is provided to deliver fluid to a patient fluid tubing set (not shown) connected to luer connector 438 using either single flow or dual flow fluid delivery processes. This design allows the user to easily unwind the tubing 432 and spike the bulk fluid source, by removing the spike from the oval portion 420. In addition, the oval portion 420 includes clips 422 on a top surface 424 thereof to secure patient tubing (not shown), spikes, or a priming tube to the oval portion 420. The patient tubing, spikes, or priming tube can also be reattached to the clips 422 after a fluid injection procedure for easy disposal. More specifically, after an injection, the user can grasp the oval portion 420 of the frame 400 to remove all of the disposables (i.e., the syringes 30, the frame 400, the tubing 432, the spikes, and the patient tubing) at once.

Various other examples for the frame for engagement with syringes or caps thereof having an active valve have been envisioned. These examples generally include a similar structure of a first engagement member for engaging a first syringe or cap and at least a second engagement member for engaging at least a second syringe or cap with a body structure connected therebetween and a hinged or uniform support structure extending from the body portion. While the support structure has been described as being oval-shaped, this is not to be construed as limiting, as the support structure may have any suitable shape. In some instances, the fastening mechanism for connecting two halves of support structure may comprise a plurality of protrusions provided on a first one of the halves configured to be engaged by a plurality of recesses or holes provided on a second one of the halves for securing the halves together to form the support structure. In another example, the first and second engagement mechanisms may each be provided with two or three latch elements to secure the syringes 30 thereto.

In some instances, the frames disclosed herein may be utilized with caps that include passive valves that do not require vertical movement of the syringes 30 in order to change the state of the valve. In such instances, the first and second engagement members may have a generally O-shaped structure configured to enclose and fit over the neck or finish of the syringes 30 before assembling the caps 50. The first and second engagement members would be constrained around the finish of the syringes 30 such that the syringes do not move during an injection.

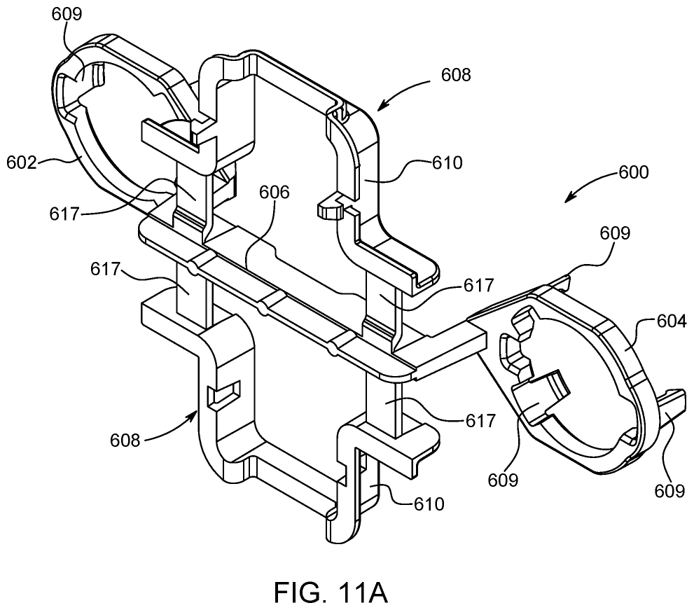

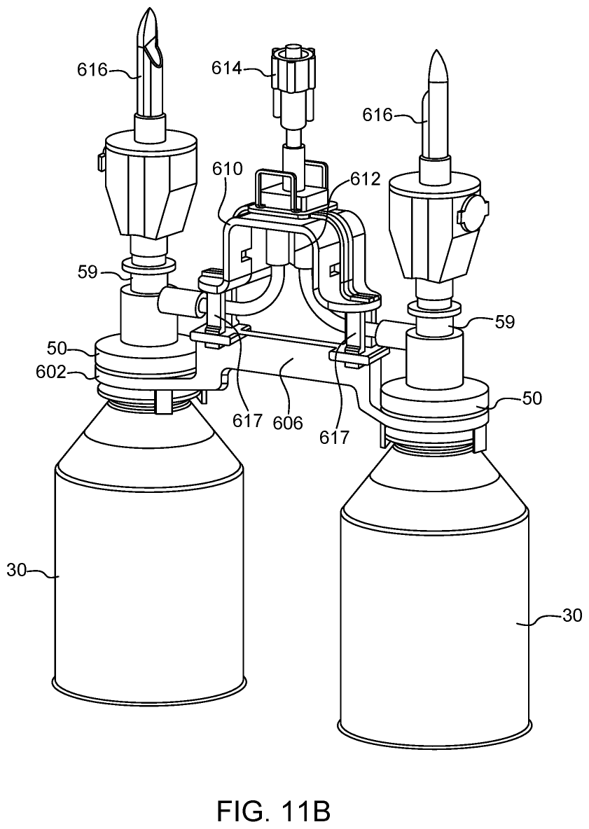

With reference to FIGS. 11A and 11B, another example of a frame 600 according to the present disclosure comprises a first engagement member 602 configured to secure at least a portion of a first syringe 30, a second engagement member 604 configured to secure at least a portion of a second syringe 30, and a body structure 606 connected to the first engagement member 602 and the second engagement member 604. According to certain embodiments, the first and second engagement members 602, 604 may each include a plurality of clipping members 609 for engaging a retention flange 43 at a distal end of the syringe to hold the at least one syringe within the engagement members. As shown in FIGS. 11A and 11B, the illustrated frame 600 may be designed for use with an active syringe valve As with the previously described embodiments, the body structure 606 is sized and shaped to index the first and second syringes 30 with the fluid injector 10 such that the first and second syringes 30 are held in an orientation and distance relative to each other for centering and insertion to the proper depth within the pressure jackets 16 of the fluid injector 10.

The frame 600 further comprises a support structure 608 extending from the body structure 606. The support structure 608 comprises a U-shaped portion 610 configured to secure a valve assembly 612 and tubing in fluid communication with outlets of the first and second syringes 30. According to certain embodiments, the support structure 608 may be hingedly attached to the body structure 606, for example by support beams 617 as described in certain previous embodiments, and may have an open form (shown in FIG. 11A, for example for ease of manufacturing) and a closed form (FIG. 11B) when the frame 600 is in its form as used during an injection protocol. The valve assembly 612 is provided to deliver fluid to a patient fluid tubing set (not shown) connected to connector assembly 614 from one of the syringes 30 while preventing fluid from being delivered to the patient fluid tubing set from the other syringe 30 or for allowing dual flow of a selected ratio of a first fluid and a second fluid from the first syringe 30 and second syringe 30, respectively. In various embodiments, the U-shaped portion 610 also serves as a handle for the user to easily grab the entire assembly and place into the injector and may include one or more gripping surfaces (not shown) thereon.

In addition, spikes 616 may be provided for filling syringes 30. In some examples, the spikes 616 may be directly connected to the caps 50 provided on syringes 30 (see FIG. 11B). In other examples, the spikes 616 may be incorporated into the support structure 608 of the frame 600. In some examples, the spikes 616 may be in direct fluid communication with the fluid inlet path 59 of the cap 50 as shown in FIG. 11B for example as a sliding cap as described herein or a non-sliding cap. Alternatively, spikes 616 may be provided in fluid communication with syringes 30 via tubing or a manifold incorporated into frame 600.

While in many examples the various components of the system are manufactured as separate components, in some instances it may be desirable to have the frame, support structure, and syringes manufactured integrally.

In certain embodiments of the present disclosure, pressure jackets 16 having a one-piece design are described, where the syringe 30 is inserted into the pressure jacket 16 from the distal end of the pressure jacket 16 (see FIG. 1). The discharge neck 42 of the syringe 30 may protrude from the distal end of the pressure jacket 16 such that the syringe may be connected to fluid lines leading to the patient and the distal end of the fluid injector may include a syringe retention element or feature, as described in PCT Application No. PCT/US2019/050293, that provides a support surface for the distal frusto-conical distal end 40 of the syringe. During an injection procedure as the piston moves in the distal direction, an exterior wall of the syringe expands against an interior wall of the pressure jacket as the pressurized fluid presses against the inner surfaces of the syringe. Additionally, the syringe may experience axial movement during a high pressure injection due to the proximal and distal movement of the piston acting on the syringe. In certain embodiments, axial movement of the syringe may be undesirable and may lead to inaccurate volume delivery.

Accordingly, in one example of the present disclosure, the frame may further incorporate one or more pressure jacket caps that interact with and reversibly lock to the distal end of corresponding pressure jackets. Various embodiments of pressure jacket cap designs that may be utilized with the various embodiments of the frames described herein are further described in U.S. Application Publication Nos. 2017/033619 and 2017/0333624, the disclosures of which are incorporated herein by reference.

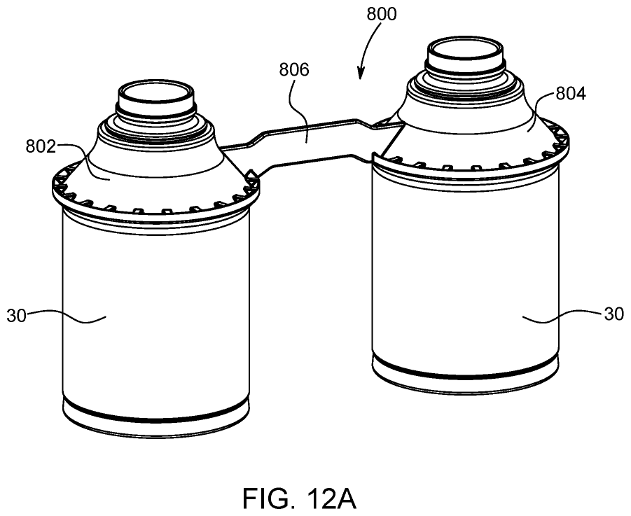

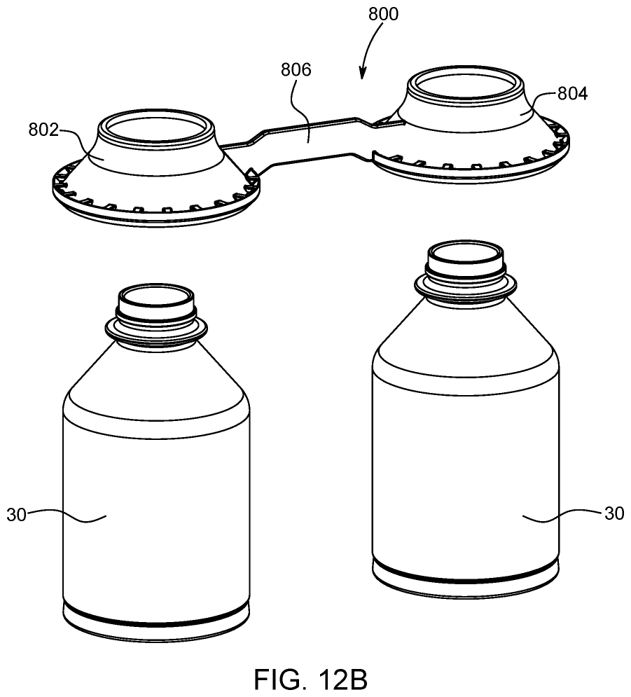

A non-limiting example of a frame 800 incorporating pressure jacket caps is illustrated in FIGS. 12A and 12B. The frame 800 comprises a first pressure jacket cap 802 and a second pressure jacket cap 804. The first and second pressure jacket caps 802, 804 each comprise a frusto-conical structure that engages and surrounds a frusto-conical distal end 40 of each of a first syringe 30 and a second syringe 30. The frame 800 further includes a body structure 806 connected to the first pressure jacket cap 802 and the second pressure jacket cap 804. As with previously described embodiments, the body structure 806 is sized and shaped to index the first and second syringes 30 with the fluid injector 10 such that the first and second syringes 30 are held in an orientation and distance relative to each other for insertion at a correct depth and centering into pressure jackets 16 of the fluid injector 10. The first and second pressure jacket caps 802, 804 may attach, for example by a click fit or by adhesion (adhesive or laser welding) to the distal end 40 of the first and second syringes 30, respectively. Alternatively, the first and second pressure jacket caps 802, 804 may be releasably attached to the distal end 40 of the first and second syringes 30 for example by a temporary or light adhesive, or a friction fit, so that the frame 800 may be removed from the syringes 30 and used with new syringes in subsequent injection procedures. As shown in FIGS. 12A and 12B, frame 800 may comprise a pair of pressure jacket caps 802, 804 that each attach to the distal end 40 of a pair of syringe 30. The first and second pressure jacket caps 802, 804 may include one or more engagement features for removable engagement with corresponding features on the distal end of a first and second pressure jacket 16.

One advantage of a frame 800 including pressure jacket caps 802, 804 may include providing a stronger support surface for the frusto-conical distal end 40 of a rolling diaphragm syringe 30. For example, due to injection pressures applied to fluid within the syringe and the thin nature of the rolling diaphragm material, the lack of support for the frusto-conical distal end 40 of the rolling diaphragm may require thicker walls at frusto-conical distal end 40 of the syringe to resist the applied pressures. In embodiments as frame 800, pressure jacket caps 802, 804 may provide the necessary thickness and support to the frusto-conical distal end 40 of the syringe so the walls of the frusto-conical distal end may have a thinner cross-section, resulting in in material and shipping savings for the syringe.

According to further embodiments, the frame 800 may incorporate one or more spikes in fluid communication with the interior volume of a syringe 30 attached to the frame, for example by a tubing set connecting the spike to a syringe cap, which may be part of the frame 800, or by a fluid manifold that is incorporated into or associated with the frame 800. Embodiments may further comprise one or more bulk fluid sources that may be removably attached to the frame and configured to move from a first, non-spiked configuration to a second, spiked configuration, for example by a movable or hinged clip structure that allows the bulk fluid source to invert and engage the spike on the frame 800 and allow fluid communication between the interior of the bulk fluid source and the interior of the syringe 30, so that the syringe 30 may be filled with fluid from the bulk fluid source. Alternatively, the bulk fluid source may remain substantially stationary and the spike may be configured to move into an engagement position to spike the bulk fluid source and allow fluid communication between the interior of the bulk fluid source and the interior of the syringe, so that the syringe 30 may be filled with fluid from the bulk fluid source. The bulk fluid sources may be supplied with the syringes and frame 800, for example as part of a packaged kit or alternatively, the user may attach a commercially available bulk fluid source to the frame 800 prior to use with the injector. It should be noted that while such embodiments are illustrated in the context of use with frame 800, it is understood that similar arrangements including spikes and clips for bulk fluid containers may be incorporated into any of the frame embodiments described herein without deviating from the scope of the present disclosure. Bulk fluid sources and containers described herein may generally contain a medical fluid, such as an imaging contrast agent or a flushing fluid, as described herein. While certain spikes in the various attached drawings are shown without corresponding spike caps, it is understood that spiking members of the present disclosure are generally provided with spike caps to maintain sterility of the system and prevent inadvertent injury to the user.

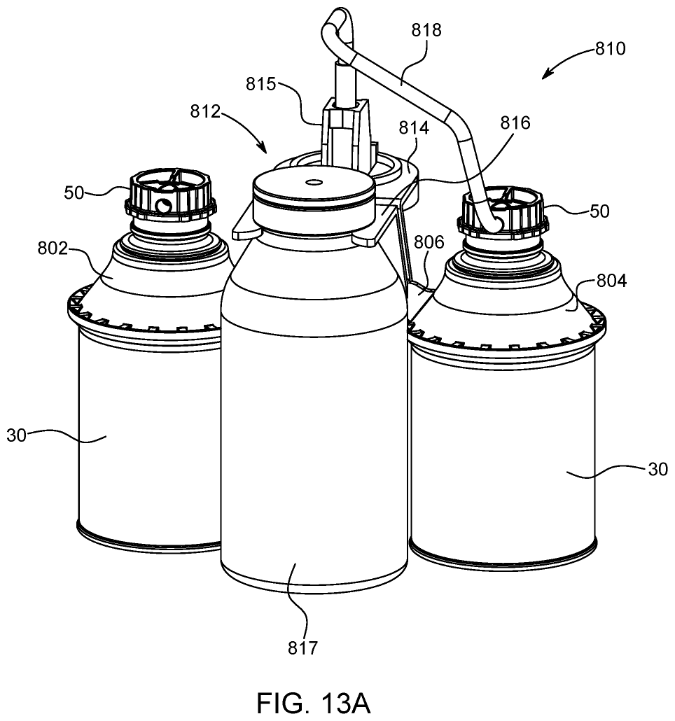

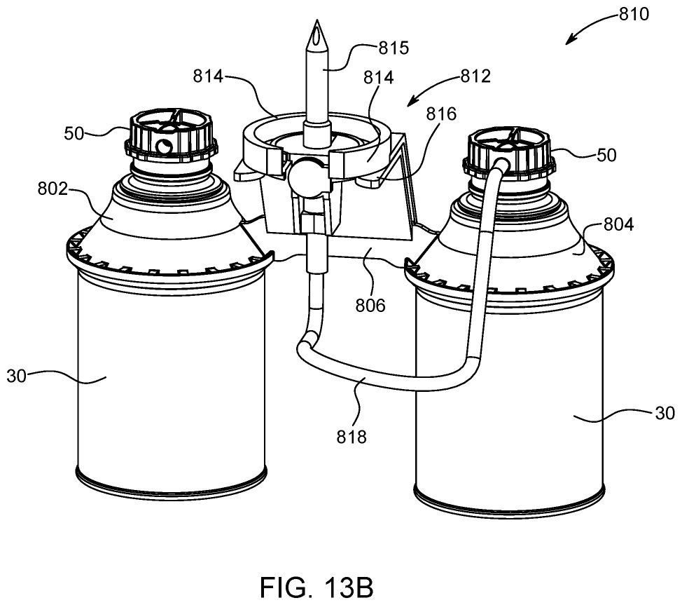

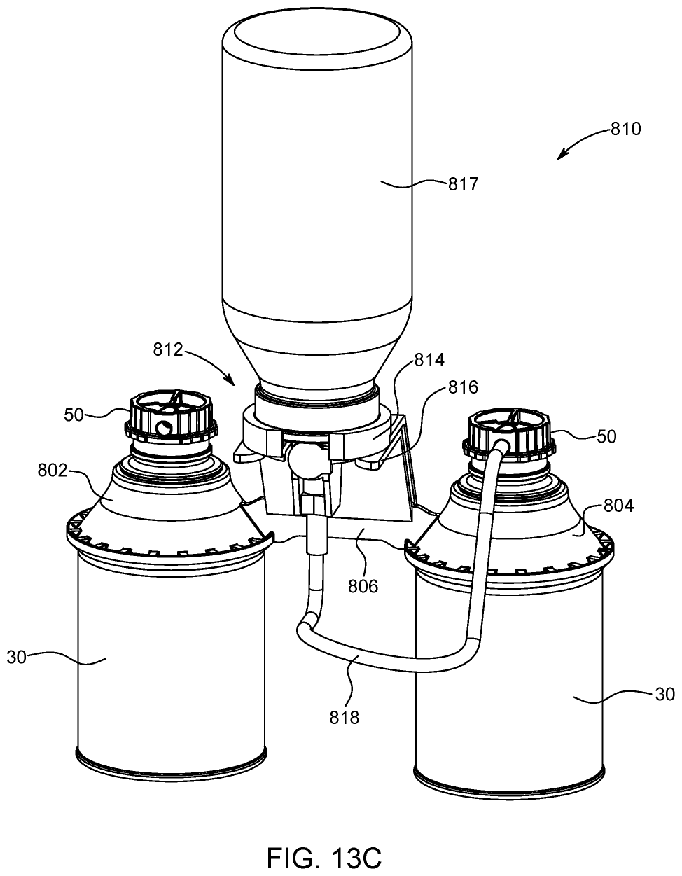

With reference to FIGS. 13A to 13C, an embodiment of a frame 810 is provided similar to frame 800 further comprising a clip structure 812 connected to and extending from the body structure 806 (see FIG. 13A). The clip structure 812 may comprise a first clip 814 extending from a first side of the body structure 806 for securing a spike 815 thereto and a second clip 816 extending from a second side of the body structure 806 for securing a bulk fluid source 817 thereto. The spike 815 may be provided in fluid communication with one of the first and second syringes 30 via tubing 818 connected to the syringe cap 50. Alternatively, the spike 815 may be provided in fluid communication with one of the first and second syringes 30 via a manifold incorporated into the frame 810.

In the present embodiment, first clip 814 may be hingedly connected to the second clip 816 such that, after the bulk fluid source 817 is removed from the second clip 816, the first clip 814 and the spike 815 may be rotated relative to the second clip 816 to provide the spike 815 in an exposed position after removal of the spike cap (see FIG. 13B). The bulk fluid source 817 may then be fluidly connected to the spike 815 by inserting the spike 815 through a septum on the cap of the bulk fluid source 817 to fill one of the first and second syringes 30 with fluid from the bulk fluid source 817 with movement of the corresponding piston associated with the syringe (see FIG. 13C).

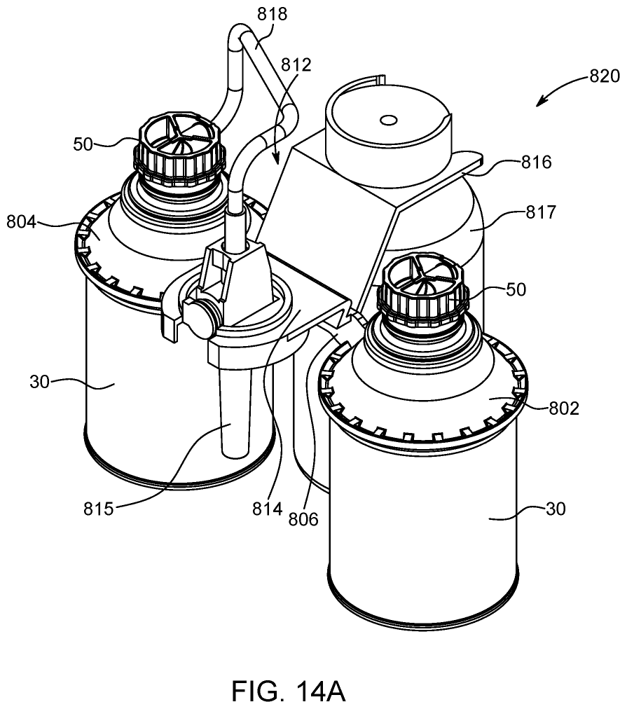

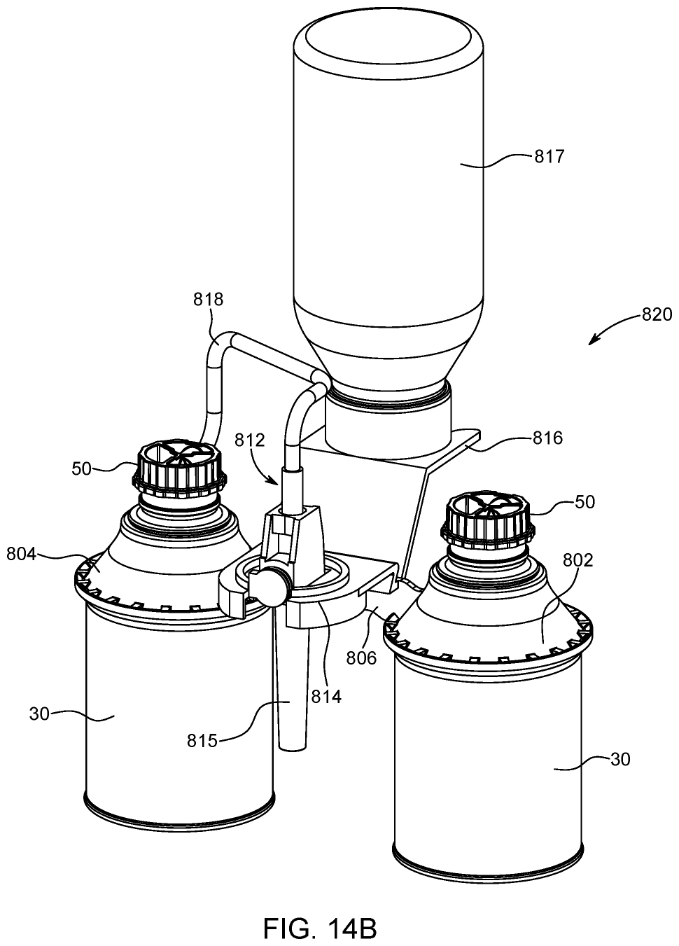

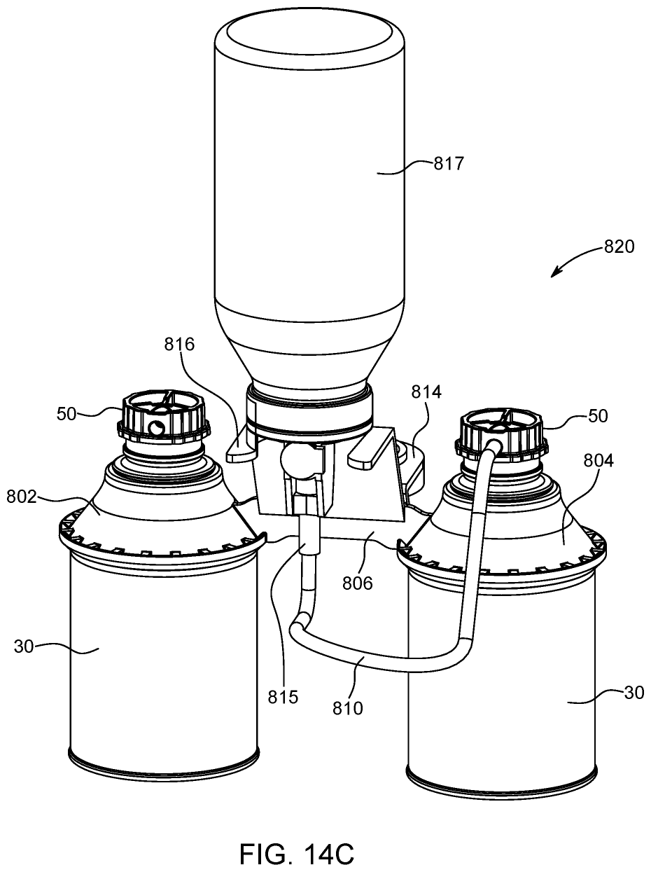

With reference to FIGS. 14A to 14C, another embodiment of frame 820 is provided similar to frame 810 except for the function of the clip structure 812 and location of certain frame components. Frame 820 may be provided to an end user as shown in FIG. 14A or an end user may use a commercially available bulk fluid source 817. Frame 820 comprises a clip structure 812 connected to and extending from the body structure. The clip structure 812 comprises a first clip 814 extending from a first side of the body structure 806 for securing a spike 815 thereto and a second clip 816 extending from a second side of the body structure 806 for securing a bulk fluid source 817 thereto. The spike 815 is provided in fluid communication with one of the first and second syringes 30 via tubing 818 connected to the syringe cap 50. In use, the bulk fluid source 817 is removed from the second clip 816 and reattached to the second clip 816 in an inverted, delivery position (see FIG. 14B). Thereafter, the spike 815 is removed from the first clip 814 and fluidly connected to the bulk fluid source 817 by insertion of the spike 815 through a septum of bulk fluid source 817 to fill one of the first and second syringes 30 with fluid from the bulk fluid source 817 by movement of the corresponding piston associated with the syringe (see FIG. 14C).

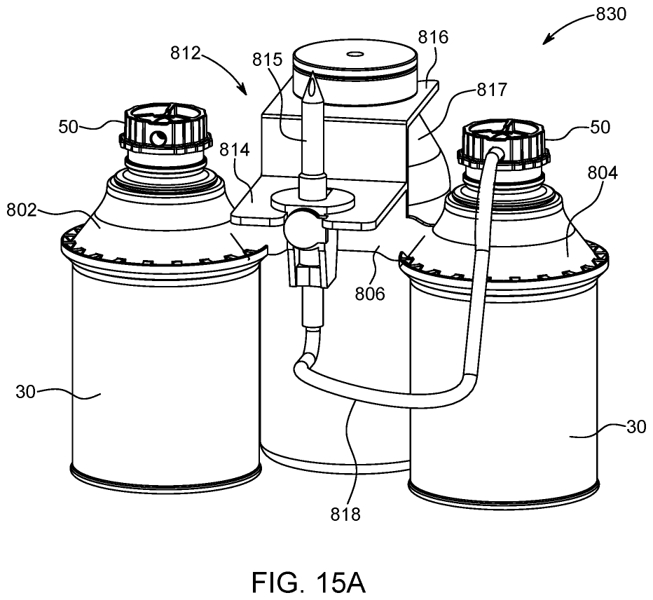

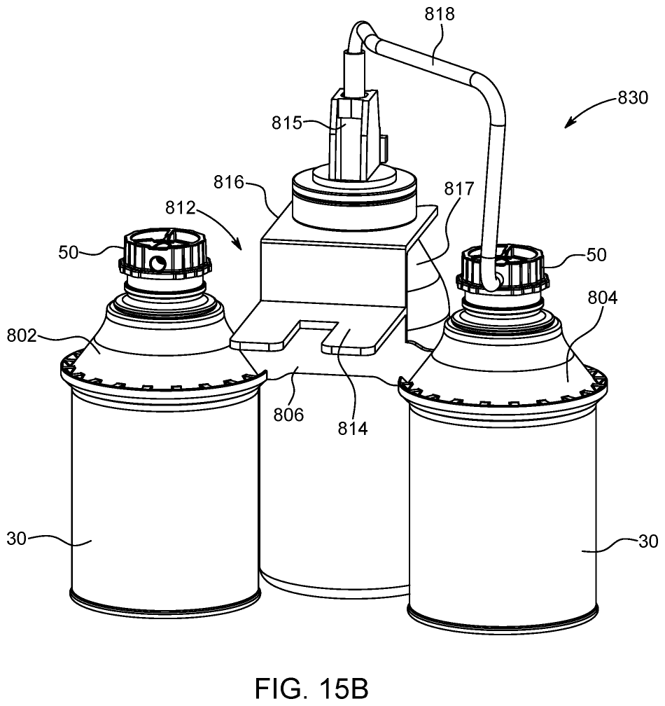

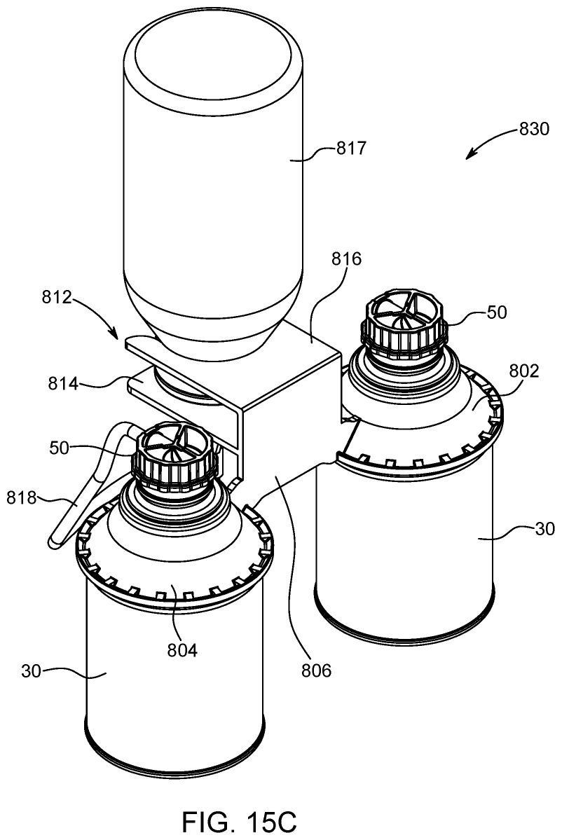

With reference to FIGS. 15A to 15C, another embodiment of frame 830 is provided similar to frame 810 except for the function of the clip structure 812 and location of certain frame components. Frame 820 may be provided to an end user as shown in FIG. 15A or an end user may use a commercially available bulk fluid source 817. Frame 820 comprises a clip structure 812 connected to and extending from the body structure. The clip structure 812 comprises a first clip 814 extending from a first side of the body structure 806 for securing a vertically upwards facing spike 815 thereto and a second clip 816 extending from a second side of the body structure 806 for securing a bulk fluid source 817 thereto. The spike 815 is provided in fluid communication with one of the first and second syringes 30 via tubing 818 connected to the syringe cap 50.

The first clip 814 is hingedly connected to the second clip 816 such that, after the spike 815 is removed from the first clip 814 and provided in fluid communication with the bulk fluid source 817 by insertion of the spike 815 through a septum of bulk fluid source 817 (see FIG. 15B), the second clip 816 and the bulk fluid source 817 are rotated relative to the first clip 814 to provide the bulk fluid source 817 and spike 815 combination in an inverted position to fill one of the first and second syringes 30 with fluid from the bulk fluid source 817 movement of the corresponding piston associated with the syringe (see FIG. 15C).

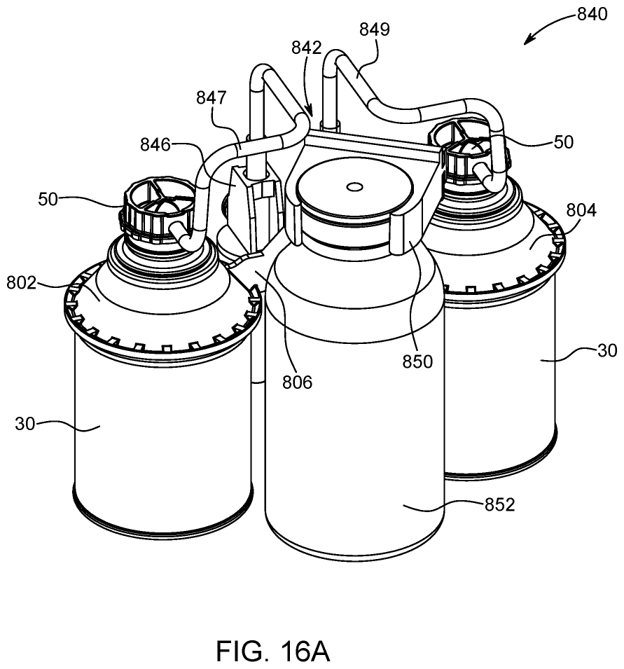

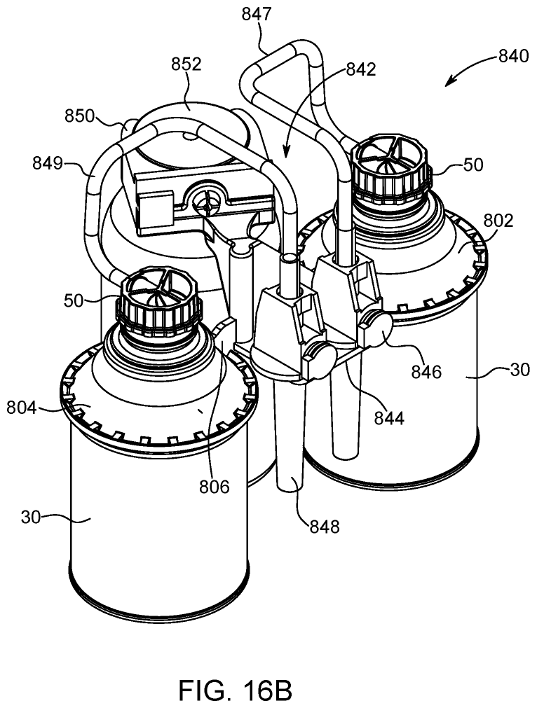

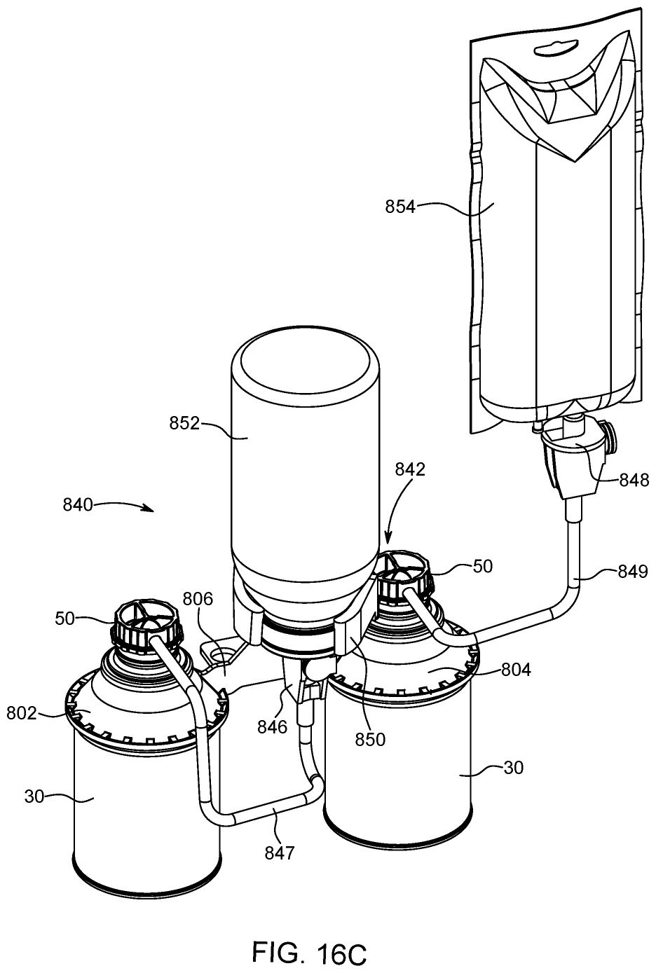

With reference to FIGS. 16A to 16C, another embodiment of frame 840 is provided similar to frame 810 except that it comprises a dual spike clip structure 842 connected to and extending from the body structure 806. Frame 840 may be provided to an end user as shown in FIGS. 16A, 16B or an end user may use a commercially available bulk fluid source 852. The clip structure 842 comprises a first clip 844 extending from a first side of the body structure 806 for securing a first spike 846 provided in fluid communication with a first syringe 30 via tubing 847 and a first cap 50 and a second spike 848 provided in fluid communication with a second syringe 30 via tubing 849 and a second cap 50. The clip structure 842 further comprises a second clip 850 extending from a second side of the body structure 806 for securing a first bulk fluid source 852, such as a bulk contrast bottle, thereto.

In operation, the first spike 846 is removed from the first clip 844 and fluidly connected to the first bulk fluid source 852 by insertion of the spike 846 through a septum of bulk fluid source 852. Thereafter, the first bulk fluid source 852 is removed from the second clip 850 and reattached to the second clip 850 in an inverted position to fill the syringe 30 with fluid from the bulk fluid source 852 movement of the corresponding piston associated with the first syringe 30. In addition, the second spike 848 is removed from the first clip 844 and fluidly connected to a second bulk fluid source 854, such as a saline bag, by insertion of the spike 846 through a septum of bulk fluid source 852 to fill the second syringe 30 with fluid from the bulk fluid source 854 movement of the corresponding piston associated with the second syringe 30 (see FIG. 16C).

According to certain aspects of the above-described embodiments, frames 810, 820, 830, and 840 may be manufactured with syringes 30 and the bulk fluid source 817 attached thereto and shipped to an end user so that the end user may engage the syringe/frame/bulk fluid source combination with the injector, spike the bulk fluid source, and fill the syringes in a sequence. Alternatively, the user may use any commercially available bulk fluid source 817 and attach it to the frames 810, 820, 830, and 840 having the first and second syringes 30 attached thereto.

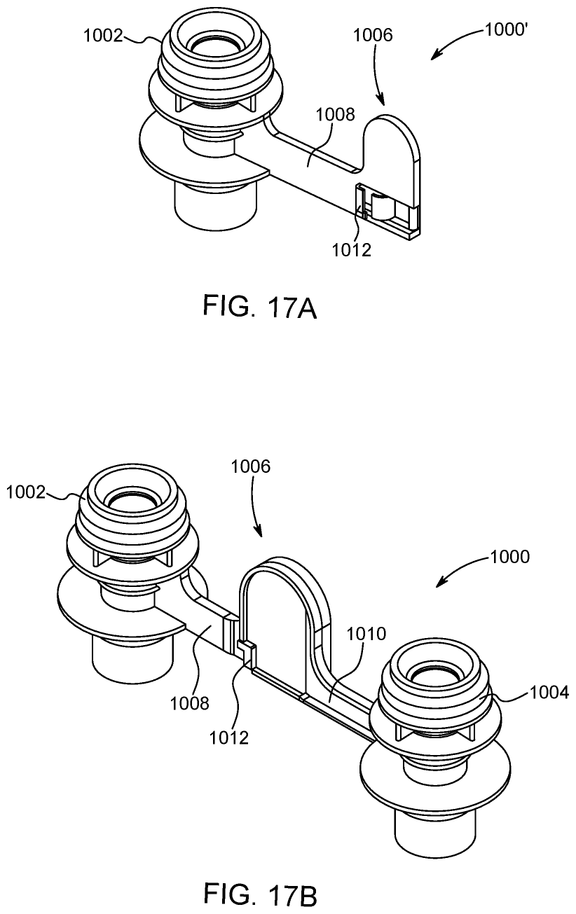

According to other embodiments, the frame may comprise separate syringe caps that may be provided and used individually (see FIG. 17A), for example for a single fluid injection procedures using a single syringe or may be snapped or otherwise connected with a second syringe cap to provide two frame connected syringes (see FIG. 17B), for example for a dual fluid injection using a pair of syringes. Each frame sub-unit may attach to a cap or distal end of a syringe, for example by a plurality of clip members (not shown in FIGS. 17A and 17B) for engaging a retention flange 43 at a distal end of the syringe and may have an engagement portion configured for interacting and connecting with the other frame sub-unit. According to single syringe cap embodiments the single frame sub-unit may include one or more frame surfaces to center and control the insertion depth of the syringe within the pressure jackets so that they are at a proper depth and centering for engaging a piston and may be held in place by one or more syringe retention members on a distal end of an injector (see, e.g., PCT Application No. PCT/US2019/050293). According to embodiments which include two frame sub-units, the frame may be used to space the two syringes apart the desired distance for engaging pressure jackets in the syringe ports of the injector and controlling the insertion depth and centering of the syringe within the pressure jackets, when the frame sub-units are engaged. The engagement mechanism may be designed to either permanently or temporarily snap the two frame sub-units together. In certain embodiments, the syringes may be shipped with each corresponding frame sub-unit and may be snapped together by an end user prior to engagement with the fluid injector, for example when a dual fluid injection is desired, or may be used separately in a single syringe injection procedure. Alternatively, the syringes may be provided in either a single fluid injection configuration having only one syringe and one frame sub-unit, or may be provided in a dual fluid injection configuration where two syringes are provided, each having a frame sub-unit that is snapped or otherwise engaged with the other frame sub-unit or may be provided in a disengaged configuration where the end user connects the frame sub-units together before engaging the syringe with the injector. The frame sub-units may be color-coded according to the fluid that is to be used in the syringe. In certain embodiments, the two frame sub-units may have identical structures where the features of the complimentary engagement mechanism are identical or mirror images so that a single mold or mold design may be used to manufacture the first and second sub-units.

With reference to FIGS. 17A and 17B, according to an aspect of the present disclosure, a frame 1000 comprises a single first syringe cap 1002 or a combination of a first syringe cap 1002 and a second syringe cap 1004. As illustrated in FIG. 17A, for single fluid injection procedures a user may use only a single syringe 30 within the injector 10 to inject an imaging contrast solution. In such embodiments, a single cap comprising a first syringe cap 1002 having a frame may be desired. According to one embodiment, a frame may be provided that can be used for a single syringe 1000′ and with a dual syringe system 1000.

With reference to a single syringe frame embodiment 1000′ in FIG. 17A, the frame 1000 may include first syringe cap 1002 configured to be positioned within the distal discharge neck 42 of a first syringe 30. The frame 1000′ further includes a body structure 1006. The body structure 1006 comprises a sub-unit portion 1008 extending from the first syringe cap 1002. The lateral end of sub-unit portion 1008 may include an engagement mechanism 1012 for complimentary engagement with a second sub-unit 1010 to convert the single syringe frame 1000′ to dual syringe frame embodiment 1000 shown in FIG. 17B. This allows for manufacture of a single sub-unit that may be used for both the single syringe frame 1000′ and the dual syringe frame embodiment 1000. Alternatively, the lateral end of sub-unit portion 1008 may not include engagement mechanism 1012 for a frame 1000′ that is only used for a single syringe injection protocol.

With reference to dual syringe frame embodiment 1000 in FIG. 17B, the frame 1000 may include first and second syringe caps 1002, 1004 configured to be positioned within the distal discharge neck 42 of each of a first syringe 30 and a second syringe 30. The frame 1000 further includes a body structure 1006. The body structure 1006 comprises a first sub-unit portion 1008 extending from the first syringe cap 1002 and a second sub-unit portion 1010 extending from the second syringe cap 1004. The first syringe cap 1002 and the first sub-unit portion 1008 of the body structure 1006 is connected to the second syringe cap 1004 and a second sub-unit portion 1010 of the body structure 1006 by an engagement mechanism 1012 provided between the first portion sub-unit 1008 of the body structure 1006 and the second sub-unit portion 1010 of the body structure 1006. The engaged sub-unit portions 1008 and 1010 form at least a portion of the combined body structure 1006. The engagement mechanism 1012 may be provided to permanently connect the first sub-unit portion 1008 of the body structure 1006 and the second sub-unit portion 1010 of the body structure 1006. Alternatively, the engagement mechanism 1012 may be configured to removably connect the first sub-unit portion 1008 of the body structure 1006 and the second sub-unit portion 1010 of the body structure 1006. Various different examples of engagement mechanisms to connect the sub-unit portions 1008 and 1010 may be utilized.

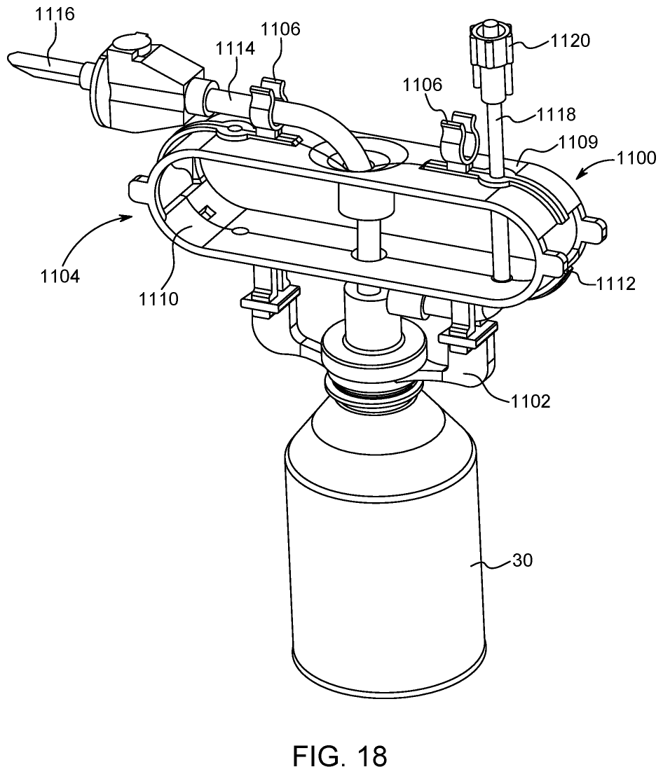

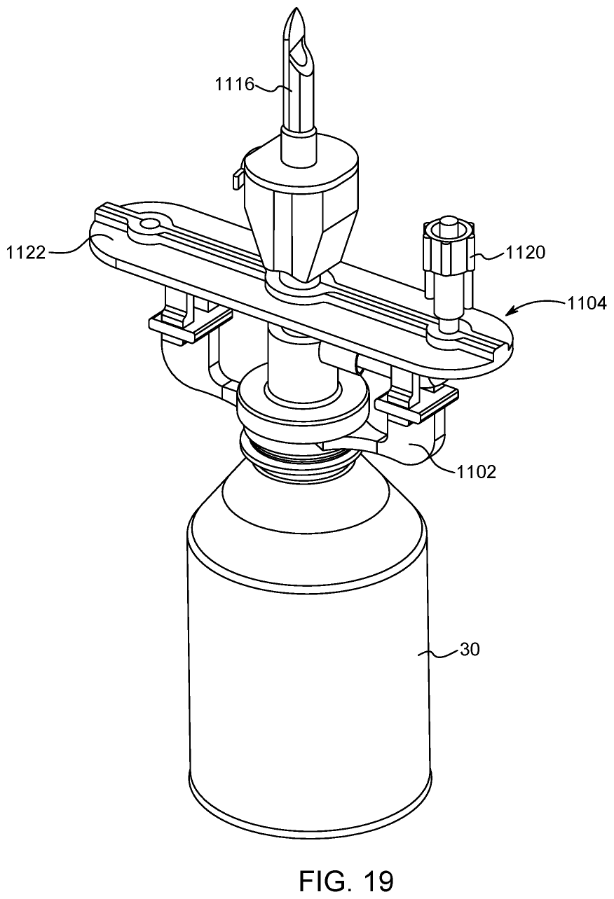

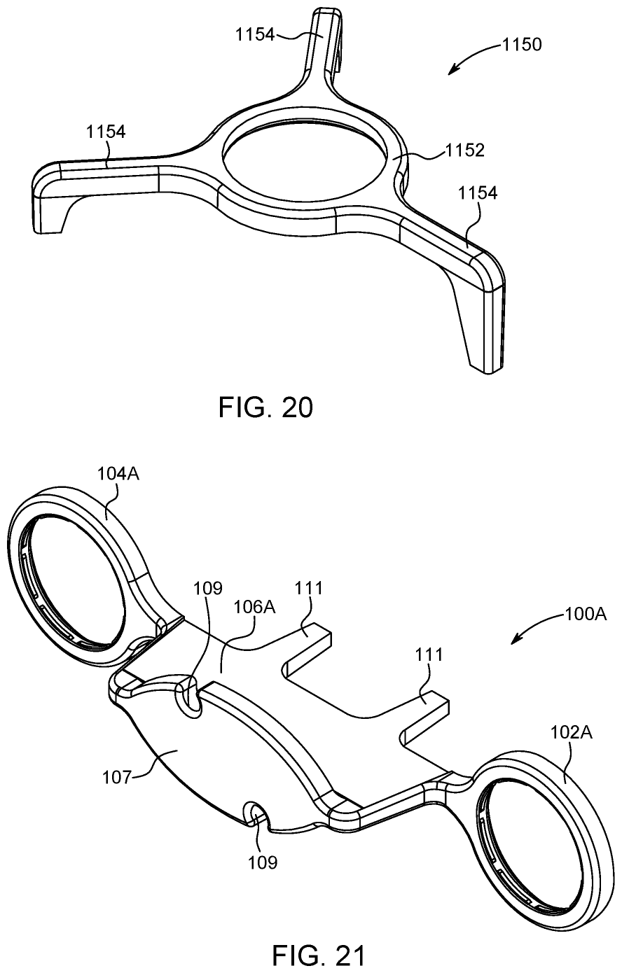

According to other embodiments, the frames may be designed with support structures for use with single syringe kits, which optionally can accommodate fill tubing (see FIG. 18) and/or a fluid manifold containing versions (see FIG. 19), as well as a version intended to solely position a single syringe at the correct vertical position within pressure jacket 16 of the injector 10 (see, FIG. 20). These frames may be similar to the dual syringe kits described above, in that the frame is used to position the syringe 30 within the injector 10 and can be used for tubing, bulk fluid source, and spike management.

Specifically, with reference to FIG. 18, an embodiment of a frame 1100 comprising a body structure 1102 positioned at a distal end 40 of a syringe 30 is illustrated. The body structure 1102 is configured to index the syringe 30 with the fluid injector 10 such that the syringe 30 is held in a proper vertical position within pressure jacket 16 of the fluid injector 10 for engaging a piston and conducting a fluid injection procedure. The body structure 1102 includes a proximal surface for indexing and locating the syringe/frame assembly relative to the pressure jacket 16. The frame 1100 may further include a support structure 1104 extending from the body structure 1102. The support structure 1104 may have an appropriate shape for managing various components used during a fluid injection procedure. In addition, the support structure 1104 may serve as a handle for the user to easily grab the entire assembly and place it into the injector. The support structure 1104 may further comprise one or more clips 1106 on a surface 1108 thereof where the clips are configured to secure tubing, spikes, and other components to the support structure 1104.

As described according to other embodiments above, support structure 1104 may be molded as a first half 1110 and second half 1112 with the first half 1110 and the second half 1112 being hingedly connected the body structure 1102 such that the first half 1110 and the second half 1112 can be connected together by snapping the first half 1110 and the second half 1112 together with a suitable fastening mechanism. The fastening mechanism may be any suitable latch, clip, or snap. This design allows for ease of molding and allows the packaging form factor to be small and compact.

The support structure 1104 may be configured to secure and manage 1) tubing 1114 used to fill the syringes 30; 2) one or more spikes 1116 used to connect the syringes 30 to a bulk fluid source; 3) a priming tube for use during a priming operation (not shown), and 4) tubing 1118 used to deliver fluid to a patient fluid tubing set (not shown) connected to connector 1120.

With reference to another embodiment illustrated in FIG. 19, the support structure 1104 and tubing features 1114, 1118 may be eliminated and replaced with a manifold 1122 that provides the spike 1116 and connector 1120 in fluid communication with the syringe 30 through a fluid pathway in manifold 1122.

With reference to FIG. 20, an embodiment is illustrated where frame 1150 is provided for a single syringe 30 for use in a single fluid injection protocol to ensure that the syringe 30 is provided with the proper vertical and centered orientation and depth when positioned within a pressure jacket 16 for engagement with a piston associated with an injector 10. The frame 1150 may include a circular engagement structure 1152 for engaging the discharge neck 42 of the syringe 30 or surface of a retention flange 43 at a distal end of the syringe and a plurality of indexing beams 1154 positioned around the circumference of the engagement structure 1152. The plurality of indexing beams 1154 are configured to engage at least a portion of a distal end of the injector 10 or pressure jacket 16 to provide the syringe 30 at the proper centered and vertical orientation.

Any of the embodiments of the frames described herein may be provided with at least one identification component provided thereon, for example of the body structure or support structure, configured to be read by a processor of the fluid injector to provide information to the fluid injector regarding the syringe assembly or bulk fluid sources. The identification component may be an RFID tag, a barcode, a QR code, or any other suitable indicia or device capable of storing information and/or having information written thereto. In some embodiments, the information may comprise at least one of information related to the syringes, information related to an injection procedure, information related to the patient, or any combination thereof. Additional details regarding the use of such identification components with frames along with other frame concepts is provided in PCT International Patent Application No. PCT/US2019/050296 incorporated herein by this reference.

In addition, the various frames described hereinabove may provide the user with an indication that the syringes provided with the frame have been used. For example, if the tubing is unwound or has fluid therein, it is easy for a user to detect that the system has been already been used and should be exchanged for a new syringe assembly. Still further, in some aspects of the present disclosure the frame may be color coded. For example, one end of the frame may be blue and the other end may be green to correspond with a color coded spike fluidly connected to a syringe on the end for filling with saline and contrast, respectively. This could also be orientation specific to ensure that a particular syringe is located on the appropriate side, for example a prefilled or empty syringe set.

According to certain embodiments where the syringes are preassembled with the fill tubing and associated spikes, the frame may include a corresponding indicator (e.g., color, texture, etc.) on the frame that correlates with the spike and/or tube set, to make it easier for the user to identify the appropriate fluid source, without having to be orientation specific. Further, the frame can also be used to provide space for writing or attaching patient information, as well as an orientation feature for a camera or other optical detector to read or detect the patient information. Still further, the injector 10 may offer sensor alignment in which an indicator on the injector 10 reads when the syringes 30 supported by the frame are appropriately installed, to allow the activation and closing of a syringe retention device of the injector 10 that is configured to hold the syringes and frame securely in the injector 10.

According to various embodiments, the frame may be used as a fluid indicator for the tubing set by placing features on the frame, such as molded edges or printed markings that appear differently when there is fluid within the tubing versus air. The frames may also provide an indication to a user that the syringes supported thereby are intended for multi-patient use or single patient use. For example, an assembly without a connecter on the end of the valve assembly and just the patient tubing can be designated for single use, while a connector at the end of the valve assembly can be designated for multi-patient use. In addition, the frames can also have indications such as colored features to determine multi-patient versus single patient syringe sets.

While specific embodiments of the device of the present disclosure have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the device of the present disclosure which is to be given the full breadth of the claims appended and any and all equivalents thereof.

1. A frame for engaging at least two fluid containers, the frame comprising:
a first engagement member configured to secure at least a portion of one of the at least two fluid containers;
a second engagement member configured to secure at least a portion of another one of the at least two fluid containers; and
a body structure connected to the first engagement member and the second engagement member,
wherein the body structure is sized and shaped to index the at least two fluid containers with a powered injector system such that the at least two fluid containers are held in an orientation and distance relative to each other for insertion into the powered injector system.

2. The frame of claim 1, wherein the at least two fluid containers are syringes.

3. (canceled)

4. The frame of claim 1, wherein the first engagement member and the second engagement member are generally C-shaped and are configured to engage a retention flange provided on a distal end of each of the fluid container.

5. (canceled)

6. The frame of claim 1, wherein the body structure comprises a first leg having a first end extending vertically from the first engagement member and a second end, a second leg having a first end extending vertically from the second engagement member and a second end, and a body member connected between the second end of the first leg and the second end of the second leg.

7. The frame of claim 6, wherein lengths of the first leg and the second leg are selected to provide a proper vertical orientation of the at least two fluid containers when positioned within the powered injector system.

8. The frame of claim 1, wherein the body structure is configured as a handle for carrying the at least two fluid containers.

9. The frame of claim 1, further comprising a support structure extending from the body structure.

10. The frame of claim 9, wherein the support structure is configured to secure a valve assembly in fluid communication with outlets of the at least two fluid containers, the valve assembly configured to deliver fluid to a patient fluid tubing set from one of the at least two fluid containers while preventing fluid from being delivered to the patient fluid tubing set from others of the at least two fluid containers or to deliver a selected ratio of at least two fluids to a patient fluid tubing set from the at least two fluid containers when a motive force is applied to each piston of the powered injector system associated with the at least two fluid containers.

11. The frame of claim 9, wherein the support structure is configured to support and manage at least one of tubing used to fill the at least two fluid containers; spikes used to connect the at least two fluid containers to a bulk fluid source; patient tubing; a priming tube; or any combination thereof.

12. (canceled)

13. (canceled)

14. The frame of claim 9, wherein the support structure is molded as a first half and second half with the first half and the second half are connected to the body structure via a hinge mechanism such that the first half and the second half can be connected together to form the support structure by closing the hinge mechanism.

15. (canceled)

16. The frame of claim 1, wherein the first engagement structure engages a fluid outlet end of one of the at least two fluid containers and the second engagement structure engages a fluid outlet end of another one of the at least two fluid containers in a manner to allow limited proximal and distal movement of the at least two fluid containers during a fluid filling and a fluid injection procedure, respectively.

17. The frame of claim 1, wherein the first engagement structure engages a fluid outlet end of one of the at least two fluid containers and the second engagement structure engages a fluid outlet end of another one of the at least two fluid containers in a manner to prevent proximal and distal movement of the at least two fluid containers during a fluid filling and a fluid injection procedure.

18-23. (canceled)

24. The frame of claim 1, further comprising at least one clip structure connected to and extending from the body structure.

25. The frame of claim 24, wherein the at least one clip structure comprises a first clip structure and a first clip extending from a first side of the body structure for securing a first spike, provided in fluid communication with at least one of the at least two fluid containers, thereto and a second clip structure and a second clip extending from a second side of the body structure for securing a bulk fluid source thereto.

26-32. (canceled)

33. The frame of claim 1, wherein the body structure comprises at least one identification component provided thereon configured to be read by the powered injector system to provide information to the powered injector system.

34. (canceled)

35. A syringe system for a powered injector system, the syringe system comprising:
a first syringe;
a second syringe; and
a frame for engaging the first syringe and the second syringe, the frame comprising:
a first engagement member configured to secure at least a portion of the first syringe;
a second engagement member configured to secure at least a portion of the second syringe; and
a body structure connected to the first engagement member and the second engagement member,
wherein the body structure is sized and shaped to index the first syringe and the second syringe with the powered injector system such that the first syringe and second syringe are held in an orientation and distance relative to each other for insertion into the powered injector system.

36-38. (canceled)

39. The syringe system of claim 35, wherein the body structure comprises a first leg having a first end extending vertically from the first engagement member and a second end, a second leg having a first end extending vertically from the second engagement member and a second end, and a body member connected between the second end of the first leg and the second end of the second leg.

40. (canceled)

41. (canceled)

42. The syringe system of claim 35, further comprising a support structure extending from the body structure.

43. (canceled)

44. The syringe system of claim 42, wherein the support structure is configured to support and manage at least one of: tubing used to fill the first syringe and the second syringe; spikes used to connect the first syringe and the second syringe to a bulk fluid source; patient tubing; a priming tube; or any combination thereof.

45-59. (canceled)

60. A syringe for a powered injector system, the syringe comprising:
a proximal end, a distal end, and a cylindrical sidewall between the proximal end and the distal end defining an interior volume for retaining a medical fluid therein;
a discharge nozzle at the distal end;
a piston engagement feature located on one of plunger slidably associated with the syringe and a proximal end wall of the syringe, the piston engagement feature configured for releasably engaging a piston of the powered injector system; and
a frame positioned at the distal end of the syringe,
wherein the frame is configured to index the syringe with the power injector system such that the syringe is held in a proper vertical position within the powered injector system for conducting a fluid injection procedure.

61-72. (canceled)
